(19) United States
(12) Patent Application Publication
(10) Pub. No.: US 2002/0025510 A1
(43) Pub. Date: Feb. 28, 2002

(54) Screening methods based on superactivated alpha V beta 3 integrin

(76) Inventors: Alex Y. Strongin, San Diego, CA (US); Elena I. Deryugina, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/916,658

(22) Filed: Jul. 26, 2001

Related U.S. Application Data
(60) Provisional application No. 60/220,706, filed on Jul. 26, 2000.

(51) Int. Cl. C12Q 1/00; C12Q 1/68; C12N 9/64

(52) U.S. Cl. 435/4; 435/6; 435/226

(57) ABSTRACT

The present invention is directed to a method of identifying an inhibitor or enhancer of αᵥβ₃ activity by contacting superactivated αᵥβ₃ integrin with one or more molecules; and assaying an αᵥβ₃ integrin activity, where reduced αᵥβ₃ activity identifies an inhibitor of αᵥβ₃ activity and where enhanced αᵥβ₃ activity identifies an enhancer of αᵥβ₃ activity. In a preferred embodiment, a cell, such as a MCF-7 breast carcinoma cell, is transfected with a nucleic acid molecule encoding a superactivated β3 variant, which can have, for example, substantially the amino acid sequence of SEQ ID NO:6 shown in FIG. 3.

### BACKGROUND OF THE INVENTION

This application is based on, and claims the benefit of, U.S. Provisional Application No. 60/220,706, filed Jul. 26, 2000,and entitled SCREENING METHODS BASED ON SUPERACTIVATED (Vβ3 INTEGRIN, and which is incorporated herein by reference.

1. Field of the Invention

The present invention relates generally to the fields of molecular medicine and anti-cancer and tissue regeneration therapeutics and, more specifically, to drugs that are inhibitors or enhancers of the function of superactivated αᵥβ₃ integrin.

2. Background Information

The concerted action of matrix metalloproteinases (MMPs) and integrins have been implicated in a variety of processes involved in tumor progression (Coussens et al., Chemistry and Bioloqy 3:895-904 (1996); Ruoslahti, Tumor Biol. 17:117-124 (1996); Varner and Cheresh Curr. Opin. Cell Biol., 8:724-730 (1996); and Cheresh, Cancer and Metastasis Rev., 10:3-10 (1991)). The membrane-type MMPs, which include MT1-MMP, MT2-MMP, MT3-MMP, MT4-MMP, MT5-MMP and MT6-MMP are distinguished from other MMPs by the existence of a C-terminal transmembrane domain that associates MT-MMPs with the lipid membrane bilayer (Seiki, APMIS 107:137-143 (1999)). In addition to directly degrading certain components of the extracellular matrix as do other membrane-type MMPs, MT1-MMP initiates the activation pathway of secretory pro-Gelatinase A, also known as pro-MMP-2, by cleaving the pro-MMP-2 polypeptide chain at Asn₆₇-Leu₆₈ (Sato et al., Nature 370:61-65 (1994); Strongin et al., J. Biol. Chem. 268:14033-14039 (1993); Strongin et al., J. Biol. Chem. 270:5331-5338 (1995)).

Integrins are cell surface receptors that are essential for adhesion and locomotion of cells through extracellular matrix substrata and tissue barriers. 1<Integrins also play a role in neovascularization during development and tumorigenesis and are upregulated in tumors such as breast carcinomas. Thus, inhibitors of integrin-mediated cell adhesion can be effective anti-cancer therapeutic agents (Strongin et al., J. Biol. Chem. 268:14033-14039 (1993) and Friedlander et al., Science 270:1500-1502 (1995)).

One of the major integrins, Uvβ37 was demonstrated to be critically involved in locomotion and metastasis of tumor cells such as breast cancer cells (Friedlander et al., Science 270:1500-1502 (1995) and Gladson et al., Am. J. Pathol. 148:1423-1434 (1996)). The main extracellular matrix ligands of the αᵥβ₃ integrin are vitronectin and fibronectin. Binding of αᵥβ₃ to vitronectin and fibronectin, which is RGD-dependent, can be inhibited with RGD peptides including cyclopeptides and αᵥβ₃ -specific function-blocking monoclonal antibodies (Strongin et al., J. Biol. Chem. 268:14033-14039 (1993); Storgard et al., J. Clin. Invest., 103:147-154 (1999); Brooks et al., Cell, 79:1157-1164 (1994); Horton, Exp. Nephrol. 7:178-184 (1999); and Craig et al., Biopolymers 37:157-175 (1995)). However, because the molecular mechanisms involved in interactions of the integrin with its ligands are poorly understood, specific inhibitors of (XVβ3 other than antibodies or RGD peptides have not been readily identified. The present invention satisfies this need by providing a novel screening method for discovery of αᵥβ₃ -inhibiting anti-cancer therapeutics, which is based on a novel, superactivated form of αᵥβ₃. The present invention provides related advantages as well.

### SUMMARY OF THE INVENTION

The present invention is directed to a method of identifying an inhibitor or enhancer of αᵥβ₃ activity by contacting superactivated αᵥβ₃ integrin with one or more molecules; and assaying an αᵥβ₃ integrin activity, where reduced αᵥβ₃ activity identifies an inhibitor of αᵥβ₃ activity and where enhanced αᵥβ₃ activity identifies an enhancer of αᵥβ₃ activity. A method of the invention is practiced by detecting an alteration in αᵥβ₃ integrin activity upon treatment of superactivated αᵥβ₃ integrin with a molecule. An αᵥβ₃ integrin activity assayed in a method of the invention can be, for example, cell adhesion activity such as vitronectin-binding activity, fibronectin-binding activity or adhesion to a function blocking αᵥβ₃ -specific antibody.

The methods of the invention can be conveniently performed as cell-based assays, in which superactivated αᵥβ₃ integrin is expressed on a cell, which can be, for example, a tumor cell or an immortalized cell and, in particular, a MCF-7 breast carcinoma cell. In one embodiment, a cell such as a MCF-7 cell is doubly transfected with a β3 encoding nucleic acid molecule and an MT1-MMP encoding nucleic acid molecule. The encoded β3 subunit can have, for example, substantially the amino acid sequence of SEQ ID NO: 2, and the encoded MT1-MMP polypeptide can have, for example, substantially the amino acid sequence of SEQ ID NO: 4. In another embodiment, a cell such as a MCF-7 cell is transfected with a nucleic acid molecule encoding a superactivated β3 variant, which can have, for example, substantially the amino acid sequence of SEQ ID NO: 6 shown in FIG. 3.

The present invention also provides a superactivated β3 variant that has substantially the amino acid sequence of a β3 subunit with a threonine analog at the equivalent of position 69 and a glutamine analog at the equivalent of position 70, where, when expressed together with an αᵥ subunit, the β3 variant forms superactivated αᵥβ₃ integrin in the absence of MT1-MMP. Such a superactivated β3 variant can contain, for example, a threonine at the equivalent of position 69 and a glutamine at the equivalent of position 70. In one embodiment, a superactivated ββ3 variant of the invention has substantially the amino acid sequence of SEQ ID NO: 6.

### BRIEF DESCRIPTION OF THE DRAWINGS

FIG. 1 shows the nucleotide (SEQ ID NO: 1) and amino acid sequence (SEQ ID NO: 2) of human β3.

FIG. 2 shows the nucleotide (SEQ ID NO: 3) and amino acid sequence (SEQ ID NO: 4) of human MT1-MMP.

FIG. 3 shows the nucleotide (SEQ ID NO: 5) and amino acid sequence (SEQ ID NO: 6) of a human β3 “double mutant.” Mutant positions are underlined. Mutations were generated by substituting A206 with C206, and substituting T209 with A209 using the corresponding nucleotide primers carrying the desired nucleotide sequence and the polymerase chain reaction. The mutant β3 integrin is distinguished from the wild-type β3 integrin by containing the amino acid substitutions Threonine69 and Glutamine70.

FIG. 4 shows expression of MT1-MMP and αᵥβ₃ in MCF7 transfected cells. Control MCF7, β3-MCF7, MT-MCF7 and β3/MT-MCF7 cells were stained with anti-αᵥβ₃ mAb LM609 (left panel) or with affinity purified rabbit anti-MT1-MMP antibodies (right panel). Open and shaded histograms show staining with corresponding control (normal rabbit IgG and murine mAb 45.6) and experimental antibodies, respectively. The x-axis represents mean fluorescence intensity; the y-axis represents cell number. Profiles are representative of several independent experiments.

FIG. 5 shows immunoblot analysis of MT1-MMP (A) and αᵥβ₃ (B) expression in MCF7 cells. Cells were surface labeled with biotin and lysed. (A) MT1-MMP was precipitated from precleared cell lysates with MT1-MMP-specific antibodies, separated by SDS-PAGE on 8% gels under reducing conditions, transferred to a membrane support and probed with avidin-peroxidase. An arrowhead points at the 62 kDa MT1-MMP band. (B) Integrins were precipitated from precleared cell lysates with mAb L230 specific to the αᵥ integrin subunit, separated by SDS-PAGE on 8% gels under non-reducing conditions, transferred to a membrane support and probed with avidin-peroxidase. Arrowheads indicate positions of the αᵥ (˜160 kDa) and β3 (90 and 95 kDa) bands. Minor 100 kDa bands in control MCF7 and MT-MCF7 cells correspond to the β5 subunit of integrin αᵥβ₅.

FIG. 6 shows MCF7 cells expressing integrin αᵥβ₃ adhere to function-blocking anti-ava3 mAb LM609. MCF7 cells (5×104 cells per well of a 96 well plate) were allowed to bind control mAb 45.6 and anti-αᵥβ₃ mAb LM609. Adherent cells were stained and absorbency (OD) was measured at 540 nm. Data are mean +/− SE from a representative experiment performed in triplicate. Control MCF7 cells are represented by closed bars; β₃-MCF7 cells are represented by open bars; MT-MCF7 cells are represented by hatched bars; and β₃/MT-MCF7 cells are represented by cross-hatched bars.

FIG. 7 shows activation of pro-MMP-2 by MCF7 cells. (A and B) Transfected cells (2×10⁵ cells per well of 24-well cluster) were incubated overnight in serum-free DMEM (0.3 ml per well). Purified pro-MMP-2 (7 ng per well, lane 1, pro-enzyme alone) was added to control MCF7, β₃-MCF7, MT-MCF7 and β₃/MT-MCF7 cells (lanes 2-5, respectively). Following overnight incubation, conditioned medium (A) and cell lysates (B) were analyzed by gelatin zymography. (C) Batimastat completely inhibits MMP-2 activation by β₃/MT-MCF7 cells. Cells were incubated in serum-free DMEM in the absence (lane 1) or presence (lanes 3 and 4) of 10 ng/ml pro-MMP-2 (lane 2, pro-enzyme alone). Batimastat was added at a final concentration of 50 μM (lane 4). Following overnight incubation, conditioned medium was analyzed by gelatin zymography. The apparent molecular masses of gelatinolytic bands corresponding to pro-MMP-2 (68 kDa), the intermediate (64 kDa), and the mature MMP-2 (62 kDa) are indicated.

FIG. 8 shows vitronectin-mediated adhesion and migration of MCF7 cells. (A) β₃-MCF7 (open circles) and β₃/MT-MCF7 (closed circles) cells (5×10⁴ cells per well of a 96 well plate) were allowed to bind vitronectin coated on plastic at 0.01-20 μg/ml. Adherent cells were stained with Crystal Violet, and absorbency (OD) was measured at 540 nm. (B-D) MCF7 cells (5×10⁴ cells per well) were plated in serum-free AIM-V medium in Transwells with the membrane undersurface coated with vitronectin at the concentrations indicated. Cells were allowed to migrate for 48 hours. Subsequently, cells that had migrated to the undersurface of the membrane were detached and counted. Data are mean +/− SE from a representative experiment performed in triplicate. (B) Migration of MCF7 cells in Transwell with the membrane undersurface coated with vitronectin at 2 μg/ml. (C) Migration of β₃-MCF7 and β₃/MT-MCF7 cells in Transwells with the membrane undersurface coated with increasing concentrations vitronectin. (D) β3-MCF7 and β3/MT-MCF7 cells were pre-incubated in AIM-V medium with and without 50 μM Batimastat. Following a 18 hour incubation, cells were plated into Transwells with the membrane pre-coated with vitronectin at 2 μg/ml. Where indicated, Batimastat was added at 100 μM to the both upper and bottom chambers of Transwells.

FIG. 9 shows adhesion of MCF7 cells to PEX. (A) MCF7 cells (5×10⁴ cells per well of a 96 well plate) were allowed to bind PEX coated at 20 μg/ml. Adherent cells were stained with Crystal Violet, and absorbency (OD) was measured at 540 nm. Data are mean +/− SE from a representative experiment performed in triplicate. (B) Control (45.6) and function-blocking mAbs specific to the αᵥ integrin subunit (L230 and L1A3) and integrin αᵥβ₃ (LM609) were used at 25 μg/ml to inhibit adhesion of β₃-MCF7 (closed bars) and β₃/MT-MCF7 (open bars) cells to PEX. Data are presented as a percentage +/− SE of maximal adherence (100%) observed with control mAb 45.6.

FIG. 10 shows immunoblot analyses of MCF7 cells. (A) β3-MCF7 (lane 1) and β₃/MT-MCF7 (lanes 2 and 3) cells were grown in the presence (+) and absence (−) of 50 μM Batimastat for 48 hours, surface labeled with biotin and lysed. Integrins were precipitated from precleared cell lysates with function-blocking mAb L230 specific to the (v integrin subunit, separated by SDS-PAGE on 8% gels under non-reducing conditions, transferred to a membrane support and probed with avidin-peroxidase. The molecular masses of 90 kDa and 95 kDa corresponding to the 3 bands are indicated. (B) Integrin αᵥβ₃ was imnunoprecipitated from lysates of surface biotinylated β₃-MCF7 and β₃/MT-MCF7 cells with control mAb 45.6 (lane 1) and function-blocking mAbs L230 and L1A3 specific to the (v integrin subunit (lane 2 and 3, respectively) and mAb LM609 specific to integrin αᵥβ₃ (lane 4). Samples were separated by SDS-PAGE on 8% gels under reducing conditions, transferred to a membrane support and probed with avidin-peroxidase. The position of the 3 band (˜105 kDa) is indicated by arrowheads.

FIG. 11 shows FACScan analysis of MCF7 breast carcinoma cells stably transfected with the double mutant β3 integrin subunit.

FIG. 12 shows migration in transwells of MCF7 breast carcinoma cells transfected with the β3 integrin subunit. (A) Migration on vitronectin. (B) Migration on fibronectin.

FIG. 13 shows adhesion to vitronectin of MCF7 breast carcinoma cells transfected with the β3 integrin subunit.

### DETAILED DESCRIPTION OF THE INVENTION

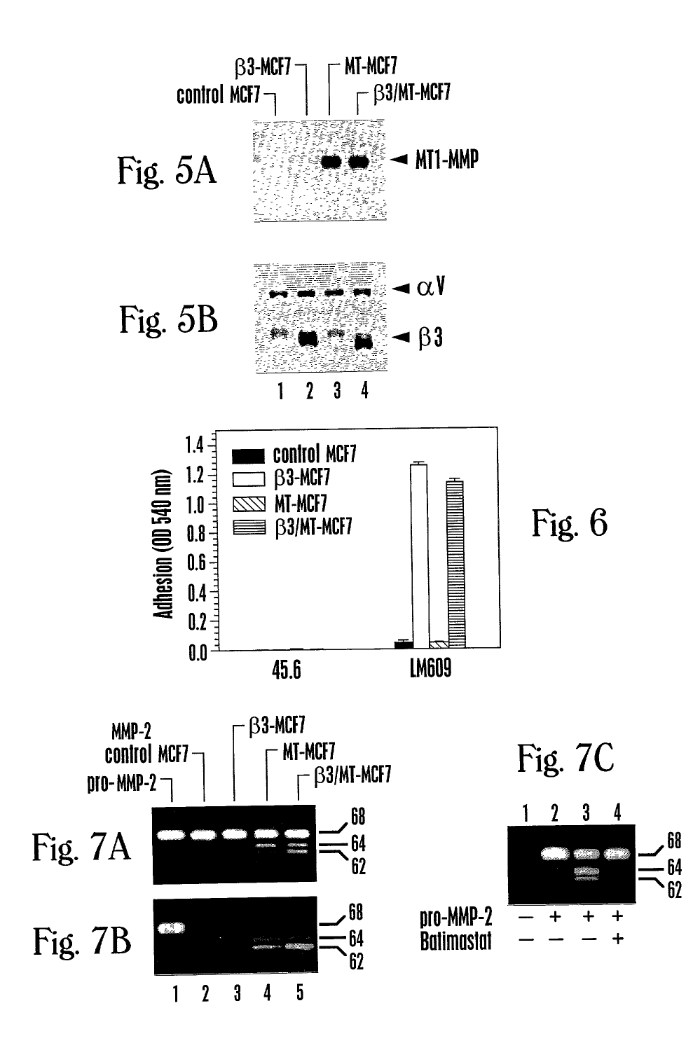

MT1-MMP and integrin αᵥβ₃ are associated with discrete regions of cell surfaces and appear to functionally cooperate in activating pro-MMP-2. As disclosed herein, parental MCF-7 cells, which express no detectable levels of pro-MMP-2, MT1-MMP or integrin αᵥβ₃ but which express substantial amounts of the αᵥ subunit, were stably transfected with either MT1-MMP, the β3 integrin subunit, or both. As shown in Example I, MT1-MMP mediates modification of the β3 subunit, resulting in a shift in molecular weight from about 95 kDa to about 90 kDa (see FIG. 5). This modification correlated with functional activation of integrin αᵥβ₃, such as increased vitronectin-mediated adhesion and cell migration. In addition, the MT1-MMP-dependent functional activation of αᵥβ₃ correlated with efficient adhesion to the recombinant C-terminal domain of MMP-2 and the generation of soluble and cell surface-associated mature MMP-2 enzyme (see Examples II to IV). As further disclosed herein, “superactivated” αᵥβ₃ integrin can be produced using a β3 variant containing two amino acid substitutions relative to the wild type β3 sequence (see Example V). The results disclosed herein provide the basis for a novel screening assay for identifying inhibitors of superactivated αᵥβ₃ integrin, which can be useful, for example, as anti-cancer or anti-angiogenic agents. Such an anti-angiogenic agent can be useful in treating any of a variety of conditions characterized by increased angiogenesis including, for example, ophthalmic disorders such as diabetic retinopathy or macular degeneration.

The present invention is directed to a method of identifying an inhibitor or enhancer of αᵥβ₃ activity by contacting superactivated αᵥβ₃ integrin with one or more molecules; and assaying an αᵥβ₃ integrin activity, where reduced αᵥβ₃ activity identifies an inhibitor of αᵥβ₃ activity and where enhanced αᵥβ₃ activity identifies an enhancer of αᵥβ₃ activity. A method of the invention is practiced by detecting an alteration in αᵥβ₃ integrin activity upon treatment of superactivated αᵥβ₃ integrin with a molecule. An αᵥβ₃ integrin activity assayed in a method of the invention can be, for example, cell adhesion activity such as vitronectin-binding activity, fibronectin-binding activity or adhesion to a function blocking αᵥβ₃ -specific antibody.

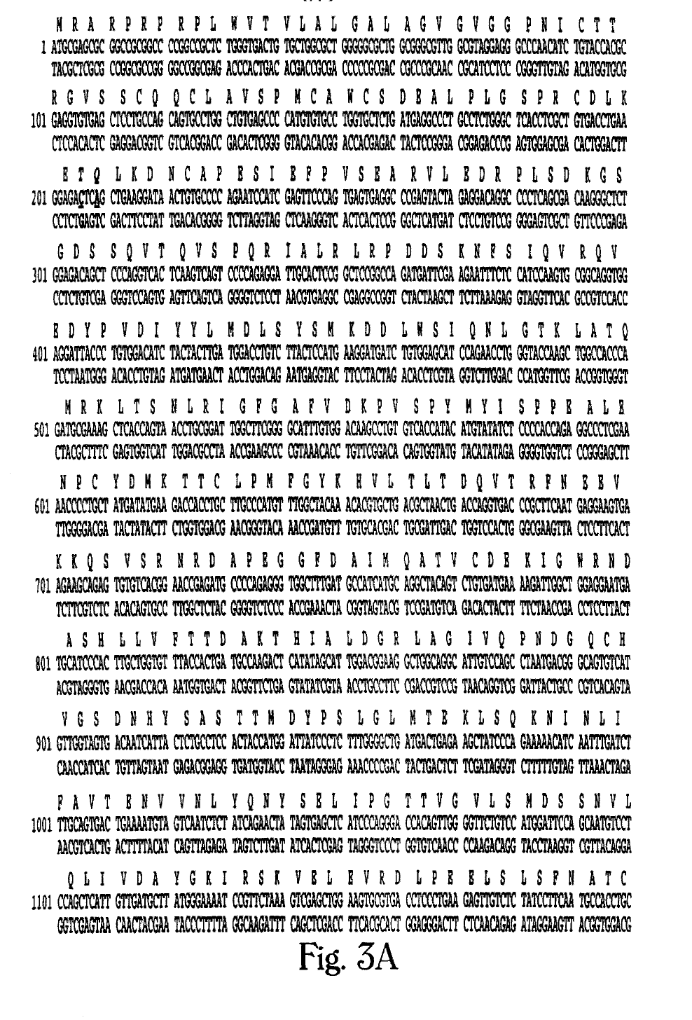

The methods of the invention rely on superactivated αᵥβ₃ integrin. As used herein, the term “superactivated αᵥβ₃ integrin” or “superactivated αᵥβ₃ ”means a form of the αᵥβ₃ integrin that is significantly more active than wild type αᵥβ₃ where wild type αᵥβ₃ contains wild type full-length αᵥ and β₃ subunits. Superactivated αᵥβ₃ is characterized, in part, in that it is functionally similar to αᵥβ₃ integrin expressed in β3/MT1-MMP MCF-7 cells. For example, the adhesion efficiency to PEX of superactivated αᵥβ₃ integrin expressed in β3/MT1-MMP MCF-7 cells was substantially greater than that of cells expressing αᵥβ₃ integrin in β3-MCF-7 cells that do not express MT1-MMP and, therefore, express wild type αᵥβ₃ . Superactivated αᵥβ₃ also can be characterized, for example, by having significantly higher vitronectin-binding efficiency than wild type αᵥβ₃ or resulting in significantly higher vitronectin-mediated or fibronectin-mediated directional cell motility when expressed, for example, in MCF7 cells. A superactivated αᵥβ₃ integrin can contain a truncated form of wild type β3 subunit having a molecular weight of about 90 kDa or can contain, for example, a constitutively activated β3 variant having substantially the amino acid sequence disclosed herein in FIG. 3 as SEQ ID NO: 6. A “superactivated” β3 subunit refers to a form of the β3 subunit which, when combined with αᵥ subunit, forms superactivated integrin.

The methods of the invention can be conveniently performed as cell-based assays, in which superactivated αᵥβ₃ integrin is expressed on a cell such as an endothelial cell or a tumor cell. In one embodiment, superactivated αᵥβ₃ integrin is expressed on a tumor cell or an immortalized cell such as a MCF-7 breast carcinoma cell. In a preferred embodiment, a cell such as a MCF-7 cell is doubly transfected with a β3-encoding nucleic acid molecule and an MT1-MMP-encoding nucleic acid molecule. The encoded β3 subunit can have, for example, substantially the amino acid sequence of SEQ ID NO: 2, and the encoded MT1-MMP polypeptide can have, for example, substantially the amino acid sequence of SEQ ID NO: 4. In another embodiment, a cell such as a MCF-7 cell is transfected with a nucleic acid molecule encoding a superactivated β3 variant, which can have, for example, substantially the amino acid sequence of SEQ ID NO: 6 shown in FIG. 3.

A cell useful in the invention is any cell capable of expressing superactivated (vβ3 integrin, including immortalized cells, tumor cells or primary cells. One skilled in the art understands that, preferably, such cells express low levels of most integrins. For example, MCF-7 breast carcinoma cells express high levels of αᵥβ₃ which can act as a donor of the αᵥ integrin, and relatively low levels of other integrins and, therefore, are particularly useful in the methods of the invention. One skilled in the art further understands that cells that express low or undetectable levels of the membrane-type matrix metalloproteinase MT1-MMP and low or undetectable levels of the β3 subunit also can be particularly useful in the invention.

Tumor cells useful in the invention include human tumor cells such as breast tumor cells, melanoma cells, colon tumor cells, prostate tumor cells, glioblastoma cells, renal carcinoma cells, neuroblastoma cells, lung cancer cells, bladder carcinoma cells, plasmacytoma cells and lymphoma cells. Such cells can be genetically engineered to express superactivated integrin as disclosed herein, for example, by double transfection with SEQ ID NO: 2 and SEQ ID NO: 4 encoding nucleic acid molecules, or by single transfection with a SEQ ID NO: 6 encoding nucleic acid molecule.

Non-tumor cells such as stromal or endothelial cells also can be useful in the invention. Such cells can be genetically engineered as disclosed above to express superactivated integrin, thereby accelerating the locomotion and adhesion of these cells to various extracellular matrix substrata and facilitating blood capillary growth and blood supply in patients. Furthermore, normal cells such as pancreatic cells capable of producing active insulin can be genetically engineered as disclosed above to express superactivated integrin in order to facilitate adhesion to the proper sites in transplantation treatment of diabetic patients.

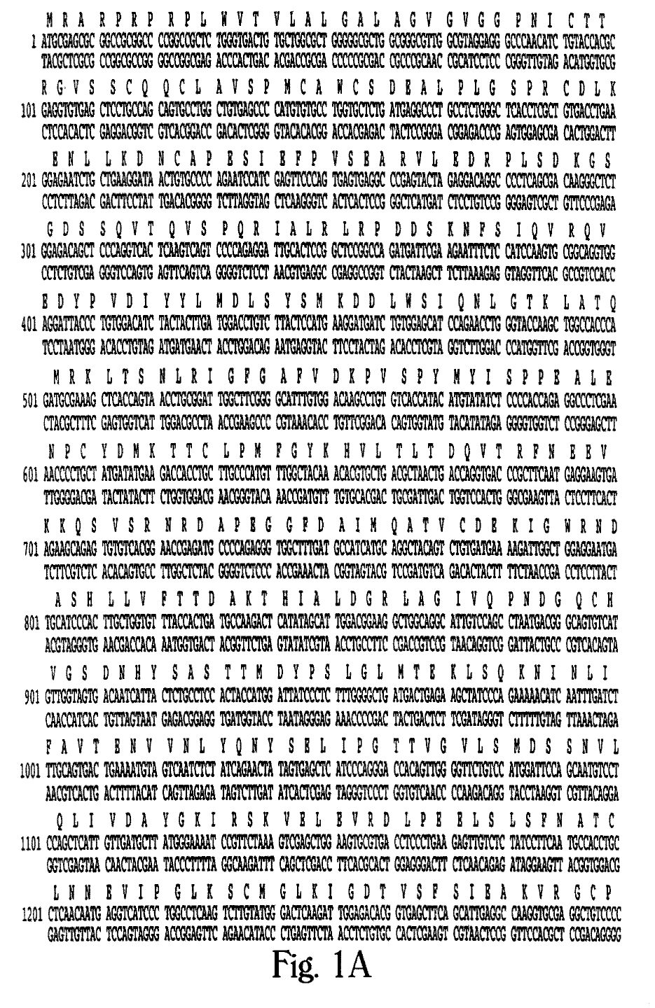

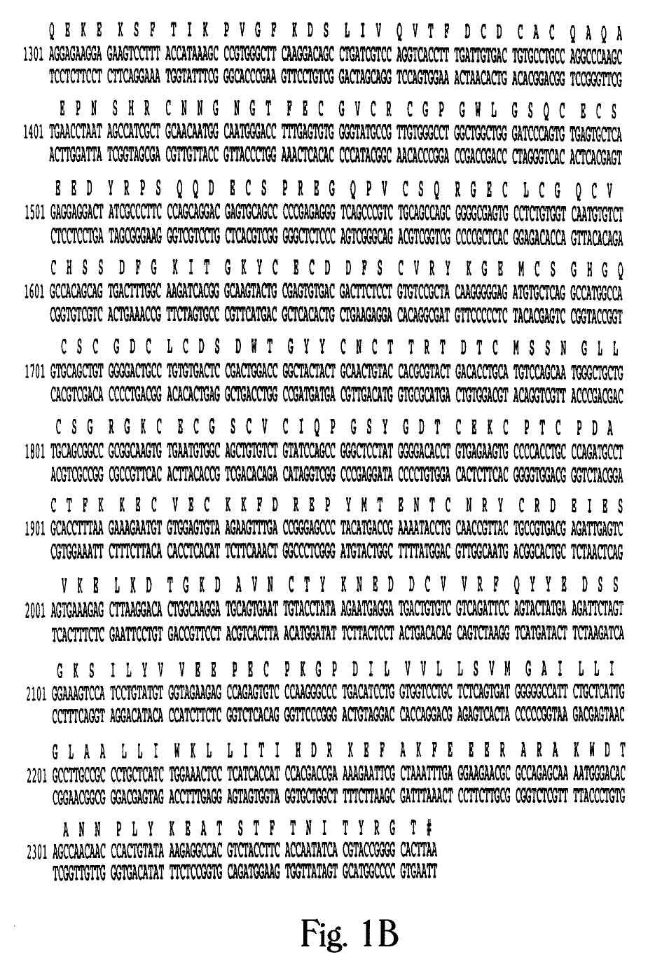

In one embodiment, a method of the invention is performed with a cell that expresses a recombinant β3 encoding nucleic acid molecule and a MT1-MMP encoding nucleic acid molecule. The β3 encoding nucleic acid molecule can encode substantially the amino acid sequence of SEQ ID NO: 2; an exemplary β3 (CD61) encoding nucleic acid molecule is provided herein as SEQ ID NO: 1 in FIG. 1 and is available as GenBank accession J02703. Additional β3 encoding nucleic acid sequences are available as GenBank accession numbers AAA52589, β05106, A26547, AAA60122, AAA35927, A60798, AAB71380, AAA52600, AAF44692, AAA67537, B36268 and AAA36121.

Integrin β3 (also known as human endothelial glycoprotein, Gβ3A, GPI11a, ITGB3, CD61 and platelet glycoprotein 3a) is the common β subunit partner of the members of the α subfamily of integrins and is generally characterized by the existence of a long extracellular domain which adheres to their ligands, and relatively short transmembrane and cytoplasmic domains that direct the integrin to the cell plasma membrane and link the integrin to the cytoskeleton of the cell, respectively. Human integrin β3 has four cysteine-rich domains, four glycosylation sites, 56 cysteine residues, and a total length of 762 amino acids with a cytoplasmic domain of 47 amino acid residues. The cysteine residues are involved in interchain disulfide bonds. Position 59 is associated with platelet-specific alloantigen HPA-1 (ZW or PL(A)). HPA1A/PL(A1) has Leu-59 and HPA-1B/PL(A2) has Pro-59.

Position 169 is associated with platelet-specific alloantigen HPA-4 (PEN or YUK). HPA-4A/PEN(A)/YUK(A) has Arg-169 and HPA-4B/PEN(B)/YUK(B) has Gln-169. HPA-4B is involved in neonatal alloimmune thrombocytopenia. Position 433 is associated with platelet-specific alloantigen MO. MO(−) has Pro-433 and MO(+) has Ala-433. MO(+) is involved in neonatal alloimmune thrombocytopenia. Position 515 is associated with platelet-specific alloantigen CA (TU). CA(−)/TU(−) has Arg-515 and CA(1)/TU(+) has Gln-515. CA(+) is involved in neonatal alloimmune thrombocytopenia.

Defects in integrin β3 are one of the causes of Glanzmann thrombastenia (GTA), an autosomal recessive disorder which is the most common inherited disease of platelets. GTA is characterized by mucocutaneous bleeding of mild to moderate severity and the inability of this integrin to recognize macromolecular or synthetic peptide ligands.

Integrin β3, in conjunction with integrin αᵥ , forms the vitronectin receptor (αᵥβ₃) This heterodimeric receptor is localized to platelets, endothelial cells, monocytes, macrophages, osteoclasts and tumor cells. The vitronectin receptor functions to mediate the adhesion of cells to vitronectin as well as a variety of extracellular matrix proteins. Receptor-protein binding is mediated by the tripeptide sequence arginine-glycine-aspartic acid (“RGD”). Activation of αᵥβ₃ can promote cellular migration and provide signals for cell proliferation and differentiation. Furthermore, upregulation of αᵥβ₃ is associated with pathological conditions such as vascular restinosis, excessive bone resorption, tumor progression, angiogenesis and macular degeneration.

Integrin β3, in conjunction with integrin α_{IIb}, also forms the fibrinogen receptor (α_{IIb}β₃), which mediates platelet aggregation. This receptor is basally inactive but can be activated by several agonists, causing it to bind fibrinogen, which then forms cross-bridges to fibrinogen receptors on adjacent cells. This receptor also binds other proteins including fibronectin, von Willebrand factor and vitronectin.

The term β3 or β3 subunit encompasses a polypeptide having the sequence of the naturally occurring human β3 (SEQ ID NO: 2) and is intended to include related polypeptides including alternatively spliced forms having substantial amino acid sequence similarity to human β3 (SEQ ID NO: 2). Such related polypeptides exhibit greater sequence similarity to human β3 than to other βintegrin subunits and include alternatively spliced forms of human β3, species homologs and isotype variants of the amino acid sequences shown in FIG. 1. A β3 polypeptide generally is characterized by an extracellular ligand binding domain, a transmembrane and a cytoplasmic domain as well as cysteine-rich domains and glycosylation sites. As used herein, the term β3 polypeptide describes polypeptides generally having an amino acid sequence with greater than 50% identity, preferably greater than 60% identity, more preferably greater than 70% identity, and can be a polypeptide having greater than 75%, 80%, 85%, 90%, 95% or greater amino acid sequence identity with human β3 (SEQ ID NO: 2).

An active fragment of a β3 polypeptide also can be useful in the invention. Such an active fragment can contain, for example, the N-terminal portion of the extracellular domain such as residues 26 to 500 of SEQ ID NO: 2, which is involved in binding the matrix substrata and defines the modified phenotype of the superactivated integrin.

As used herein, the term “substantially the amino acid sequence,” when used in reference to the β3 amino acid sequence SEQ ID NO: 2, is intended to mean the sequence shown in FIG. 1 or a similar, non-identical sequence that is considered by those skilled in the art to be a functionally equivalent amino acid sequence. For example, an amino acid sequence that has substantially the same amino acid sequence as SEQ ID NO: 2 can have one or more modifications such as amino acid additions, deletions or substitutions relative to the wild type sequence of human β3 (SEQ ID NO: 2), provided that the modified polypeptide retains substantially at least one biological activity of β3, such as substantially the ability to form an αᵥβ₃ integrin that is sufficient for cell adhesion to vitronectin or fibronectin.

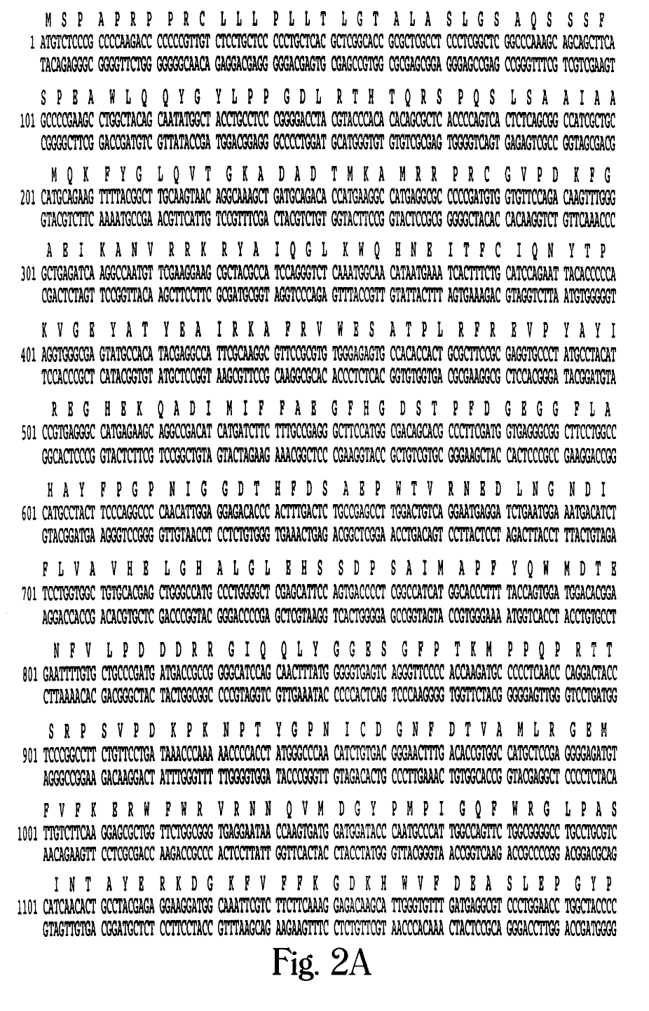

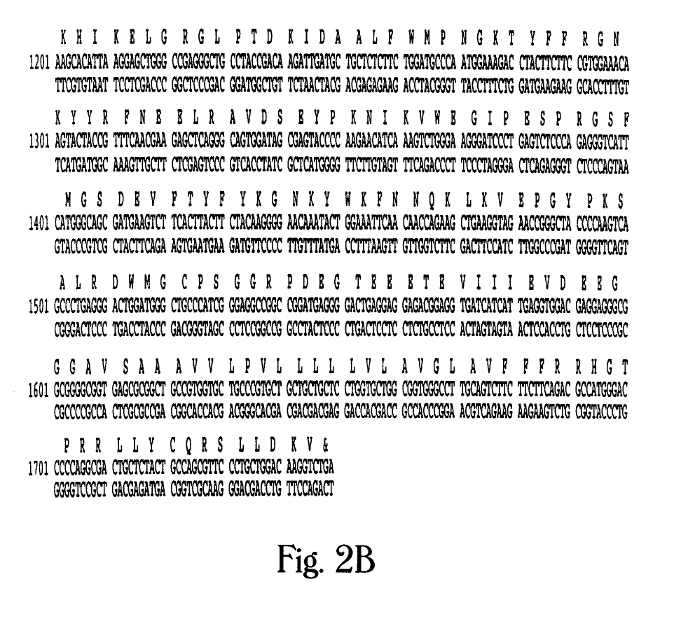

As set forth above, a method of the invention can be performed with a cell that expresses a recombinant β3 encoding nucleic acid molecule in combination with a MT1-MMP encoding nucleic acid molecule. Such an MT1-MMP encoding nucleic acid molecule can encode, for example, substantially the amino acid sequence of SEQ ID NO: 4; an exemplary MT1-MMP encoding nucleic acid molecule is provided herein as SEQ ID NO: 3 in FIG. 2 and is available as GenBank accession U641078. Additional MT1-MMP encoding nucleic acid sequences are available as GenBank accession numbers NM004995, D26512,×90925 and X83535.

MT1-MMP polypeptide chain generally has a modular domain structure and contains a signal peptide, propeptide domain, catalytic domain, hemopexin-like domain, transmembrane domain and cytoplasmic domain. The signal peptide is proteolytically removed during secretion and trafficking of MT1-MMP. The propeptide domain has a conserved unique PRCG(V/N)PD (SEQ ID NO: 7) sequence, in which the conserved cysteine links the catalytic zinc ion to maintain the latency of the MT1-MMP zymogen. At the C-terminal end of the propeptide, there is a processing sequence RX(K/R)R (SEQ ID NO: 8), which is susceptible to the cleavage by furin, a serine proteinase of the Golgi network

The catalytic domain (about 170 amino acids) contains a zinc binding motif HEXXHXXGXXH (SEQ ID NO: 9) and a conserved methionine, which forms a unique “Met-turn” structure. This domain consists of a five-stranded P-sheet, three a-helices and bridging loops. The catalytic domain has an additional structural zinc and 2-3 calcium ions which are required for the stability and expression of enzymatic activity. The C-terminal hemopexin-like domain (about 210 amino acid residues) has an ellipsoidal disk shape with a four bladed P-propeller structure. Each blade contains four antiparallel P-strands and an a-helix. The transmembrane domain anchors MT1-MMP to the cell surface, while the cytoplasmic domain links MT1-MMP to the intracellular compartment.

The term “MT1-MMP” is synonymous with “membrane type matrix metalloproteinase-l” and encompasses a polypeptide having the sequence of the naturally occurring human MT1-MMP (SEQ ID NO: 4) as well as related polypeptides having substantial amino acid sequence similarity to human MT1-MMP (SEQ ID NO: 4). Such related polypeptides exhibit greater sequence similarity to human MT1-MMP than to other matrix metalloproteinases and include alternatively spliced forms of human MT1-MMP, species homologs and isotype variants of the amino acid sequences shown in FIG. 2. As used herein, the term MT1-MMP polypeptide describes polypeptides generally having an amino acid sequence with greater than 50% identity, preferably greater than 60% identity, more preferably greater than 70% identity, and can be a polypeptide having greater than 75%, 80%, 85%, 90%, 95% or greater amino acid sequence identity with human MT1-MMP (SEQ ID NO: 4).

An active fragment of MT1-MMP also can be useful in the invention. Such an active fragment can contain, for example, the catalytic domain of MT1-MMP, which is involved in the proteolytic modification of integrin αᵥβ₃ disclosed herein. The catalytic domain of MT1-MMP starts downstream of the putative RRKR (SEQ ID NO: 10) furin-cleavage site and includes amino acid residues 112 to 280 of the MT1-MMP polypeptide chain shown as SEQ ID NO: 4.

As used herein, the term “substantially the amino acid sequence,” when used in reference to the MT1-MMP amino acid sequence SEQ ID NO: 4, is intended to mean the sequence shown in FIG. 2 or a similar, non-identical sequence that is considered by those skilled in the art to be a functionally equivalent amino acid sequence. For example, an amino acid sequence that has substantially the same amino acid sequence as SEQ ID NO: 4 can have one or more modifications such as amino acid additions, deletions or substitutions relative to the wild type sequence of human MT1-MMP (SEQ ID NO: 4), provided that the modified polypeptide retains substantially at least one biological activity of MT1-MMP, such as substantially the ability to degrade components of the extracellular matrix or cleavage of pro-MMP-2 (gelatinase A) at Asn₃₆-Leu_{37.}

The present invention also provides a superactivated β3 variant as well as methods which rely on a cell expressing a recombinant superactivated β3 variant. A superactivated β3 variant of the invention can have substantially the amino acid sequence of a β3 subunit with a threonine analog at the equivalent of position 69 and a glutamine analog at the equivalent of position 70, where, when expressed together with an αᵥ subunit, the β3 variant forms superactivated αᵥβ₃ integrin in the absence of MT1-MMP. Such a superactivated β3 variant can contain, for example, a threonine at the equivalent of position 69 and a glutamine at the equivalent of position 70. In one embodiment, a superactivated β3 variant of the invention has substantially the amino acid sequence of SEQ ID NO: 6.

A naturally occurring form of β3 has an asparagine (Asn) residue at position 69 and a leucine (Leu) residue at position 70. In one embodiment, the invention provides a superactivated β3 variant having substantially the amino acid sequence of a β3 subunit with a residue other than asparagine at the equivalent of position 69 or a residue other than leucine at the equivalent of position 70, or both, where, when expressed together with an αᵥ subunit, the β3 variant forms superactivated αᵥβ₃ integrin in the absence of MT1-MMP. Such a superactivated β3 variant can have, for example, a conservative or non-conservative amino acid substitution in place of asparagine at position 69 or a conservative or non-conservative amino acid substitution in place of leucine at position 70, or both, where, when expressed together with an a, subunit, the β3 variant forms superactivated αᵥβ₃ integrin in the absence of MT1-MMP. In one embodiment, such a superactivated β variant has a non-conservative amino acid substitution in place of asparagine at position 69 and a non-conservative amino acid substitution in place of leucine at position 70, where, when expressed together with an αᵥ subunit, the β3 variant forms superactivated UVβ3 integrin in the absence of MT1-MMP.

Conservative and non-conservative amino acid substitutions are well known in the art. Non-conservative substitutions are those in which the substituted residue has one or more dissimilar properties than the original amino acid, for example, a dissimilar size, hydrophobicity, polarity or charge. Asparagine, a polar residue containing a hydrogen acceptor, can be non-conservatively substituted, for example, with a non-polar residue such as alanine, valine, leucine, isoleucine, proline, methionine, phenylalanine or tryptophan; a positively or negatively charged residue such as aspartic acid, glutamic acid, lysine, arginine or histidine; or a polar residue that lacks a hydrogen-acceptor such as glycine, serine, threonine, cysteine or tyrosine, or an analog of any of these residues. Similarly, leucine, a non-polar residue, can be non-conservatively substituted, for example, with an uncharged, polar residue, or a negatively or positively charged residue. In particular, leucine can be non-conservatively substituted, for example, with any of the following residues: asparagine, cysteine, glutamine, glycine, serine, threonine, tyrosine, aspartic acid, glutamic acid, arginine, lysine, histidine or an analog of any of these residues, or a residue of a dissimilar size.

As used herein, the term “superactivated β3 variant” means a form of the β3 subunit, which, when expressed together with the a, subunit, forms superactivated UV3 integrin in the absence of MT1-MMP. Such a superactivated β3 variant can have, for example, substantially the amino acid sequence of SEQ ID NO: 6. A nucleic acid sequence encoding a β3 variant useful in the invention is provided herein as SEQ ID NO: 5 (see FIG. 3).

The term superactivated β3 variant is synonymous herein with β3 variant and encompasses a polypeptide having the sequence of the β3 variant disclosed herein as SEQ ID NO: 6 as well as related polypeptides having substantial amino acid sequence similarity to the human β3 variant (SEQ ID NO: 6). Such related polypeptides include superactivated β3 variants from other species as well as isotype variants of the amino acid sequences shown in FIG. 3. A superactivated β3 variant may be characterized by structural modification induced by MT1-MMP-dependent proteolytic cleavage of the β3 N-terminal part or substitution within residues corresponding to residues 60 to 70 of the 3 polypeptide SEQ ID NO: 2. As used herein, the term β3 variant describes polypeptides generally having an amino acid sequence with greater than 50% identity an encompasses polypeptides having greater than 60% identity, greater than 70% identity, or greater than 75%, 80%, 85%, 90% or 95% amino acid sequence identity with the human β3 variant shown in FIG. 3 (SEQ ID NO: 6), provided that the variant has a residue other than asparagine at the equivalent of position 69 and a residue other than leucine at the equivalent of position 70 and that the variant retains the activity of forming superactivated αᵥβ₃ integrin when expressed together with the αᵥ subunit in the absence of MT1-MMP.

In specific embodiments, the invention provides β3 variants having an amino acid sequence with greater than 50% identity, greater than 60% identity, greater than 70% identity, or greater than 75%, 80%, 85%, 90%, 95% amino acid sequence identity with the human β3 variant shown in FIG. 3 (SEQ ID NO: 6), provided that the variant retains Thr or an analog thereof at the equivalent of position 69 and retains Gln or an analog thereof at the equivalent of position 70 and that the variant retains the activity of forming superactivated αᵥβ₃ integrin when expressed together with the v subunit in the absence of MT1-MMP.

One skilled in the art understands that a threonine analog shares the biochemical properties of the amino acid threonine and generally is an uncharged polar amino acid having a hydroxyl group. Thus, a threonine analog can be, for example, serine, tyrosine or threonine. A threonine analog also can be an amino acid or mimetic that is biochemically more similar to threonine than to the amino acid at position 69 in wild type β3, asparagine.

Similarly, one skilled in the art understands that a glutamine analog shares the biochemical properties of the amino acid glutamine and generally is an uncharged polar amino acid having an amide group. Thus, a glutamine analog can be, for example, asparagine or glutamine. A glutamine analog also can be an amino acid or mimetic that is biochemically more similar to glutamine than to the amino acid at position 70 in wild type β3, leucine.

As used herein, the term “substantially the amino acid sequence,” when used in reference to the:3 variant amino acid sequence SEQ ID NO: 6, is intended to mean the sequence shown in FIG. 3 or a similar, non-identical sequence that is considered by those skilled in the art to be a functionally equivalent amino acid sequence. For example, an amino acid sequence that has substantially the same amino acid sequence as SEQ ID NO: 6 can have one or more modifications such as amino acid additions, deletions or substitutions relative to the wild type sequence of the human β3 variant disclosed herein (SEQ ID NO: 6), provided that the modified polypeptide retains the activity of forming superactivated αᵥ:3 integrin when expressed together with the αᵥ subunit in the absence of MT1-MMP.

Furthermore, a portion of the full-length β3 variant can be sufficient to form superactivated αᵥβ₃. The amino-terminal residues 26 to 500 of the β3 integrin extracellular domain is involved in binding the matrix substrata and the modifications within the N-terminal part of integrin β3 involving residues 69 and 70 of the integrin β3 sequence induce changes in the structure of the regions of the molecule downstream of this sequence that define the modified phenotype of the superactivated integrin.

In view of the above, it is understood that limited modifications can be made without destroying the biological function of a β3 polypeptide, MT1-MMP polypeptide or a β3 variant and that only a portion of the entire primary sequence can be required in order to effect activity. For example, minor modifications of human β3 (SEQ ID NO: 2), human MT1-MMP (SEQ ID NO: 4), or the human β3 variant disclosed herein as SEQ ID NO: 6 that do not destroy polypeptide activity also fall within the definition of a 3 polypeptide, MT1-MMP polypeptide or superactivated β3 variant, respectively. Also, for example, genetically engineered fragments of these polypeptides either alone or fused to heterologous proteins such as fragments or fusion proteins that retain measurable activity in, for example, a cell adhesion assay fall within the definition of the polypeptides as defined herein.

It is understood that minor modifications of primary amino acid sequence can result in polypeptides which have substantially equivalent or enhanced function as compared to the human β3 sequence set forth in FIG. 1, the human MT1-MMP sequence set forth in FIG. 2, or the β3 variant disclosed herein in FIG. 3. These modifications can be deliberate, as through site-directed mutagenesis, or can be accidental such as through mutation in hosts harboring an encoding nucleic acid. All such modified polypeptides are included in the definition of a β3 polypeptide, MT1-MMP polypeptide or superactivated β3 variant, respectively, as long as the biological function of the parent polypeptide is retained.

The methods of the invention can be used to screen a library of molecules. As used herein, the term “molecule” means any organic molecule and includes small molecule chemicals; peptides including peptidomimetics and peptoids; proteins, including antibodies and antigen-binding fragments thereof as well as non-antibody proteins; nucleic acid molecules including oligonucleotides; oligosaccharides; lipoproteins; glycolipids; and lipids. Both peptide and non-peptide molecules can be identified according to a method of the invention, as can, for example, non-antibody small molecules, including or excluding peptides. Both naturally occurring and synthetic molecules can be screened in a method of the invention. Naturally occurring molecules are a product of nature in that the groups comprising the molecule and the bonds linking the groups are produced by normal metabolic processes.

As used herein, the term library means a collection of organic molecules. Such a library can contain, for example, a plurality of diverse organic molecules or can contain various different but related organic molecules. A library of molecules can contain a few or a large number of different molecules, varying from about two to about 10¹⁵ molecules, or about 50 to about 10¹⁵ molecules, or about 1000 to about 10¹⁵, or about 10,000 to about 10¹⁵ molecules, as desired. For diverse libraries, the complexity of the library can vary such that the library covers at 5%, 10%, 20%, 30%, 40%, 50% or more of the entire pharmacophore space. For example, the DIVERSet™ chemical library (ChemBridge, San Diego, Calif.), which covers approximately 50% of the entire pharmacophore space, can be particularly useful in the methods of the invention.

Combinatorial chemical libraries are well known in the art for identification of lead compounds with pharmacological properties that can be improved by further structural optimization and can be particularly useful in the methods of the invention. The DIVERSet™ library can be particularly useful in the methods of the invention. DIVERSet™, designed for lead generation by ChemBridge and Chemical Design Ltd. (UK), is a diverse library of hand-synthesized chemical compounds for high-throughput screening. The DIVERSet™ library is a unique set of 10,000 to 50,000 drug-like, hand-synthesized small molecules, rationally pre-selected to form a “universal” library that covers the maximum pharmacophore diversity with the minimum number of compounds and which has been shown to be useful in screening assays (Komarov et al., Science 285:1733-1737 (1999) and Stockwell et al., Chem. Biol. 6:71-83 (1999)). According to Chemix software analyses (Oxford Molecular Group, Oxford, UK), the 36,000 component DIVERSet™ library is equivalent to approximately 50% of the entire pharmacophore space. The following filters were used in generating DIVERSet™: molecular weight higher then 190 and lower then 700; organic compounds and their salts containing any other atoms except: C, H ,N, O, S ,P, F, Cl, Br, I, Na, K, Ca, Mg; compounds with carbon count less then C8; compounds containing less then 2 major heteroatoms N, 0; more then 2 nitro groups; more than 6 Cl or Br or I atoms; more than 12 F atoms; more than three CF(CF₂) groups; compounds such as carbodiimides, cyanates, thiocyanates, isocyanides, acid halides and anhydrides, azides, diazoniums, N,P,S-halo, organic perchlorates, periodates, peroxides, ozonides, phosphines and phosphonium salts, disulfides pentafluorophenylhpenol and crown ethers, diazo non heterocycles, epoxides, primary halides other than F, iodiniums, alpha-haloheterocycles and compounds containing aliphatic aldehydo N-phosphorous, P-phosphorous, quarternary ammonium groups or 5 or more CN and more then six (CF₂) groups.

Similar synthetic libraries such as PRIME-Collection, Screen-Set, CNS-Set, Express-Pick and Cherry-Pick and other related combinatorial chemical libraries could be useful in the method of the invention. For example, PRIME-Collection 2000™ is a premier molecular diversity collection of 25,000 to 100,000 hand-crafted and 100% quality proven drug-like small molecules. Compounds in this collection are specially pre-selected from around 1.5 million molecules potentially available from thousands of international sources. PRIME-Collection 2000™ combines the most advanced efforts in developing compound collections that can speed medicinal chemistry efforts. SCREEN-Set, a library of 12,000 to 24,000 diverse drug-like small molecules selected for high-throughput screening primarily by integrated medicinal chemistry expertise. A related CNS-Set represents a library that includes compounds selected to generate leads that are more amenable to optimization in therapeutic areas that require both oral activity and blood brain barrier penetration.

The methods of the invention rely on assaying for reduced or enhanced αᵥβ₃ integrin activity to identify an inhibitor or enhancer of αᵥβ₃ activity. One skilled in the art understands that an “inhibitor” of αᵥβ₃ activity can reduce αᵥβ₃ activity either directly or indirectly and can be, for example, a precursor of an active compound. Similarly, an “enhancer” of αᵥβ₃ activity can increase αᵥβ₃ activity either directly or indirectly and can be, for example, a molecule which is a precursor of an active compound.

An αᵥβ₃ integrin activity to be assayed in a screening method of the invention can be, for example, a cell adhesion activity such as vitronectin-binding activity, fibronectin-binding activity, or binding to one or more extracellular matrix proteins that bind αᵥβ₃ , including collagen type I or IV, tenascin or laminin. An assay for αᵥβ₃ integrin activity also can be adhesion to a function-blocking αᵥβ₃ -specific antibody such as LM609. Various assays for αᵥβ₃ integrin activity are well known in the art and exemplified herein in Example VII.

If desired, a population of molecules can be assayed for activity en masse or in pools. For example, to identify an αᵥβ₃ inhibitor or enhancer, a population of molecules can be assayed for the ability to inhibit cell adhesion of a MCF-7 cell expressing a recombinant β3 variant; the active inhibitory population can be subdivided and the assay repeated in order to isolate the inhibitory molecule from the population. Such screening protocols, in which compounds are assayed in pools of 10, 50, 100, 200, 500 or 1000, for example, are well within the ability of those skilled in high throughput screening technology.

The following examples are intended to illustrate but not limit the present invention.

### EXAMPLE I

### Expression of MT1-MMP and Integrin αᵥβ₃ in MCF7 Cells

The effects of joint expression of MT1-MMP and integrin αᵥβ₃ on tumor cell functions were analyzed using a variant of the MCF7 breast carcinoma cell line devoid of pro-MMP-2, the β3 integrin subunit and MT1-MMP. Flow cytometry and immunoprecipitation experiments confirmed the absence of detectable levels of MT1-MMP or αᵥβ₃ in the parental MCF7 cells. Gelatin zymography, a method that can detect minute amounts of gelatinases, also failed to demonstrate the existence of pro-MMP-2 in medium conditioned by MCF7 cells.

To induce expression of MT1-MMP and integrin αᵥβ₃, MCF7 cells were stably transfected with the MT1-MMP and 3 cDNAs, respectively. The parental cells were first transfected with either the original pcDNA3-neo plasmid or the pcDNA3-neo plasmid carrying the 3 cDNA gene. Selected neo-MCF7 and β₃-MCF7 cells were then each transfected with either the original pcDNA3-zeo plasmid or the pcDNA3-zeo plasmid carrying the MT1-MMP cDNA gene. The resulting doubly transfected cell lines expressed none (control MCF7), one of each (β₃-MCF7 and MT-MCF7), or both 3 and MT1-MMP (β₃/MT-MCF7).

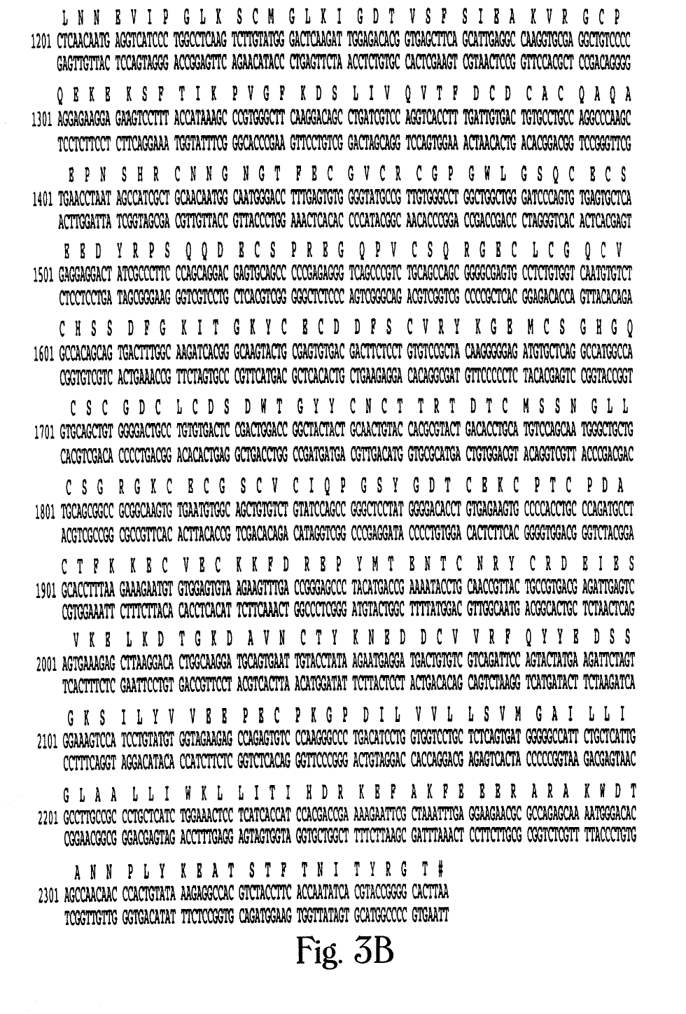

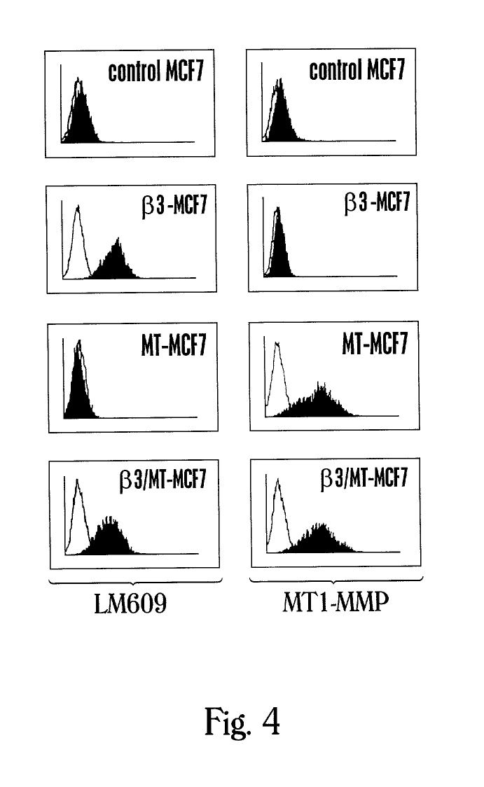

Flow cytometry and immunoprecipitation analyses confirmed high levels of surface expression of MT1-MMP and (X,3 in the corresponding MCF7 cell lines (FIGS. 1 and 2). MT1-MMP expression was observed in MT-MCF7 and β₃/MT-MCF7 cells whereas control MCF7 and β₃-MCF7 cells did not demonstrate any significant expression of cell surface MT1-MMP (FIG. 4, right panel). As shown in FIG. 5A (lanes 3 and 4), MT1-MMP specific antibodies efficiently immunoprecipitated a 62 kDa biotinylated protein from MT1-MMP-transfected MCF7 cells, which correlates well with the known molecular mass of MT1-MMP (Hoekstra et al., Curr. Med. Chem. 5:195-204 (1998) and Kerr et al., Anticancer Res. 19:959-968 (1999)).

High expression of U,β3 was observed in β₃-MCF7 and L₃/MT-MCF7 cells, both of which were transfected with the 3 integrin subunit (FIG. 4, left panel; FIG. 5B, lanes 2 and 4). In contrast, no 3 was demonstrated in control MCF7 and MT-MCF7 cells (FIG. 4, left panel; FIG. 5B, lanes 1 and 3). Relatively minor bands with an approximate molecular mass of 100 kDa (FIG. 5B), correspond to the β5 integrin subunit (Wayner et al., J. Cell Biol. 113:919-929 (1991)) and correlate with expression of αᵥβ₅ in the parental MCF7 cell line. As opposed to control MCF7 and MT-MCF7 cells, the P integrin subunit with a molecular mass of 95 kDa, characteristic of the 3 subunit (Cheresh et al., Cell 57:59-69 (1989)), was readily precipitated from β₃-MCF7 cells (FIG. 5B, lane 2). The apparent molecular mass of the β3 subunit precipitated from β₃/MT-MCF7 cells, 90 kDa, was about 5 kDa less than that of the 95 kDa β3 subunit in β₃-MCF7 cells (FIG. 5B, lane 4).

The functionality of integrin αᵥβ₃ expression in MCF7 cells was analyzed by evaluating adhesion of cells to the function-blocking αᵥβ₃-specific monoclonal antibody (mAb) LM609. No adhesion of cells was observed with control mAb 45.6, and, as expected, control MCF7 and MT-MCF7 cell lines, which both lack integrin αᵥβ₃, did not adhere to anti-αᵥβ₃ mAb LM609. However, as shown in FIG. 6, β₃-MCF7 and β₃/MT-MCF7 cell lines efficiently adhered to mAb LM609, confirming that functional integrin αᵥβ₃ was expressed in cells transfected with the β3 integrin subunit.

Proteins and antibodies were prepared as follows. Pro-MMP-2, essentially free of TIMP-2, was isolated from the conditioned medium of β2AHT2A72 cells (Strongin et al., J. Biol. Chem. 268:14033-14039 (1993)). Vitronectin was a kind gift of Dr. R. DiScipio. The recombinant C-terminal domain of MMP-2 (PEX) was isolated as a FLAG-fusion protein from the periplasmic fraction of E. coli (Strongin et al., J. Biol. Chem. 270:5331-5338 (1995)). Control monoclonal antibody (mAb) 45.6 (ATCC, Rockville, MD) and mAbs specific to the αᵥ integrin subunit, L1A3 (Deryugina et al., Hybridoma 15:279-288 (1996)) and L230 (ATCC), were purified from media conditioned by corresponding hybridoma cells. Control rabbit IgG, rabbit antibodies against MT1-MMP, and murine mAb LM609 specific to integrin αᵥβ₃ were from obtained Chemicon (Temecula, Calif.).

Gelatin zymography was performed as follows. Cells were plated at 2×10⁵ cells per well of a 24-well cluster. After overnight incubation, 0.3 ml of serum-free DMEM supplemented with purified pro-MMP-2 (10 ng/ml) was added to each well. To visualize the activity of secretory MMP-2, medium conditioned by cells for 18-24 hours was mixed 1:1 with 2×SDS sample buffer and 12 μl were loaded per lane of a precast zymography gel (Novex, San Diego, Calif.). To analyze the activity of cell-associated MMP-2, 4×10⁵ cells were lysed in 35 μl of 2×SDS sample buffer, incubated for 30 min at 37° C. and mixed 1:1 with 50% glycerol. A total of 14 μl was loaded per lane of a zymography gel. Following electrophoresis, zymography gels were incubated in 2.5% Triton X-100 and then overnight at 37° C. in the developing buffer (Novex). The bands of gelatinolytic activity were revealed after staining the gels with Coomassie Blue. When identical samples are run in replicates or different samples from a particular cell line are examined, zymography analysis demonstrates very low variability, thereby allowing quantitative as well as qualitative analysis of MMP-2 activation.

Flow cytometry was performed as follows. Cells were stained with 5 μg/ml rabbit anti-MT1-MMP antibodies or murine mAb LM609 specific to avβ3 (Deryugina et al., J. Cell Sci. 110:2473-2482 (1997); Deryugina et al., Cancer. Res. 58:3743-3750 (1998)). Cells were subsequently incubated with a FITC-conjugated F(ab′)₂ fragment of goat anti-rabbit or sheep anti-mouse IgG (Sigma, St. Louis, Mo.). Viable cells were analyzed on a FACScan flow cytometer (Becton Dickinson, Mountain View, Calif.). Population gates were set by using cells incubated with normal rabbit IgG or control murine mAb 45.6.

Immunoprecipitation and Western blotting were performed as follows. Confluent cells were surface biotinylated with 0.1 mg/ml of Sulfo-NHS-LC-Biotin (Pierce, Rockford, Ill.). Where indicated, the cells were incubated prior to biotinylation with 50 μM Batimastat for 48 hours in AIM-V medium (GibcoBRL). Cells were solubilized at 10⁷/ml in lysis buffer (50 mM n-octyl-(-D-glucopyranoside in 50 mM TBS, pH 7.4/1 mM CaCl₂/1 mM MgCl₂/1 mM PMSF/10 μg/ml leupeptin/10 μg/ml pepstatin) for one hr on ice. Insoluble material was removed by centrifugation. Supernatants were precleared for two hours at 4° C. with Protein A-Agarose (Calbiochem, La Jolla, Calif.). Precleared supernatants (100-150 μl) were incubated overnight at 4° C. with 3 μg of the indicated antibodies. The immune complexes were collected on Protein A-Agarose 15 μl of a 50% slurry) during a two hour incubation at room temperature. Following washes with washing buffer (50 mM Tris, pH 7.4/0.5 M NaCl/0.1% Tween 20), the beads were treated with 4% SDS in 125 mM Tris, pH 6.8/20% glycerol buffer, and boiled. Eluted proteins were separated by electrophoresis on a 8% acrylamide gel (Novex) under reducing or non-reducing conditions, transferred onto a Immobilon-P membrane (Millipore, Bedford, Mass.), and biotin-labeled material was visualized by using avidin conjugated to horseradish peroxidase (Sigma) and TMB/M solution containing 3,3′5,5′-tetramethylbenzidine and hydrogen peroxide (Chemicon).

### EXAMPLE II

### MT1-MMP and Integrin a9,3 Functionally Cooperate in Activation of PRO-MMP-2 to the Fully Mature Enzyme

The docking and activation of exogenous MMP-2 at tumor cell surfaces were analyzed in cells jointly expressing MT1-MMP and integrin αᵥβ₃. Pro-MMP-2, essentially free of TIMP-2, was added at 10 ng/ml in serum-free DMEM to MCF7 transfected cells (FIG. 7A and 7B, lane 1). After an overnight incubation, medium conditioned by cells was collected, and the cells were washed free of culture medium and lysed. Aliquots of conditioned medium and cell lysates were analyzed by gelatin zymography (FIGS. 4A and 4B, respectively).

As shown in FIG. 7A, lanes 2 and 3, control MCF7 and β₃-MCF7 cells did not activate exogenous 68 kDa pro-MMP-2. In contrast, MT-MCF7 cells processed the 68 kDa pro-MMP-2 to the 64 kDa activation intermediate and to some extent to the 62 kDa enzyme. Significantly higher levels of mature MMP-2, relative to those from MT-MCF7 cells, were identified in medium conditioned by β₃/MT-MCF7 cells (FIG. 7A, lanes 4 and 5). As shown by FACS (FIG. 1, right panel) and immunoprecipitation (FIG. 5A) analyses, levels of MT1-MMP expression in MT-MCF7 cells are comparable to those found in β₃/MT-MCF7 cells. These results indicate that expression of αᵥβ₃ in β₃/MT-MCF7 cells facilitates maturation of MMP-2. Incubation of β₃/MT-MCF7 cells with 50 μM Batimastat, a potent inhibitor of matrix metalloproteinases, resulted in complete inhibition of MT1-MMP and conversion of 68 kDa pro-MMP-2 to the 64 kDa intermediate (FIG. 7C, lane 4), indicating that MT1-MMP initiates the first, 68 to 64 kDa, stage of pro-MMP-2 activation while expression of αᵥβ₃ facilitates the second, 64 to 62 kDa, step of MMP-2 maturation.

Zymography analysis of cell lysates was performed to analyze the species of MMP-2 associated with MCF7 cells jointly expressing MT1-MMP and (vX3 While no gelatinolytic activity was observed in lysates of control and β₃-MCF7 cells (FIG. 7B, lanes 2 and 3, respectively), lysates of MT-MCF7 and β₃/MT-MCF7 cells demonstrated the surface bound 68 kDa proenzyme and two activated species of MMP-2, the 64 kDa intermediate and the 62 kDa fully mature enzyme (FIG. 7B, lanes 4 and 5). Of the species in β₃/MT-MCF7 cell lysates, the 62 kDa mature enzyme was the most dominant. The higher levels of MMP-2 enzyme found in β₃/MT-MCF7 cells relative to β₃-MCF7 cells indicate that expression of integrin αᵥβ₃ facilitates the MT1-MMP-induced activation of MMP-2 to the mature enzyme.

### EXAMPLE III

### MT1-MMP Mediates Functional Activation of Integrin αᵥβ₃ in MCF7 Cells

These results demonstrate that the αᵥβ₃ integrins in β₃-MCF7 and β₃/MT-MCF7 cells differ functionally in their ligand-binding capability.

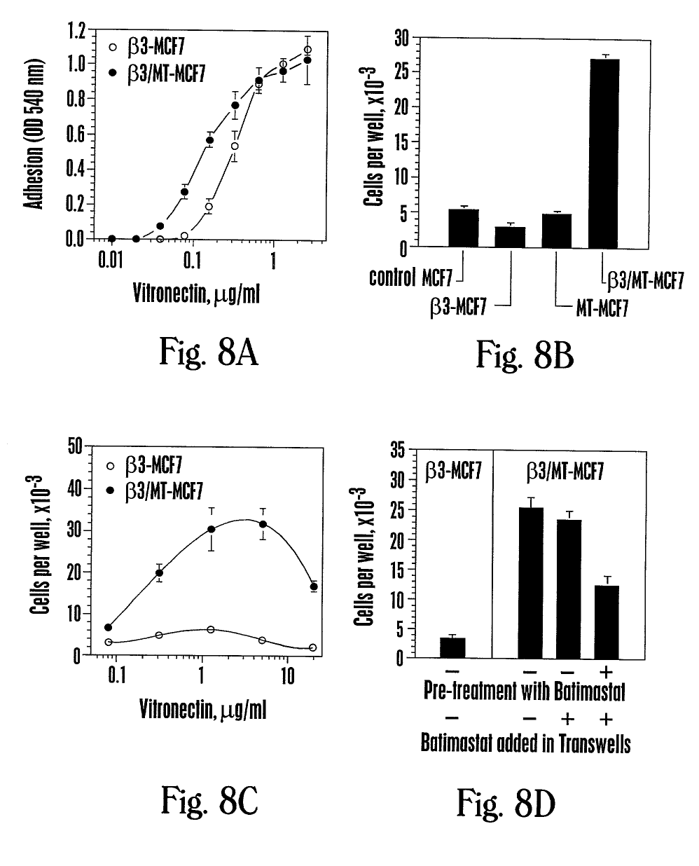

To address whether joint expression of MT1-MMP and integrin αᵥβ₃ affects the adhesion and motility of tumor cells, the vitronectin-binding efficiency of two β₃-transfected MCF7 cell lines was compared. For the cell binding assays, β₃-MCF7 and β₃/MT-MCF7 cells were allowed to adhere to increasing concentrations of vitronectin coated on plastic. Although both cell lines attached to vitronectin in a dose-dependent manner, β₃/MT-MCF7 cells demonstrated greater adhesion efficiency relative to that of β₃-MCF7 cells, especially at moderate concentrations of the ligand. As shown in FIG. 8A, 50% maximal adhesion of β₃-MCF7 cells was achieved at 5-10-fold greater coating concentration of vitronectin as compared to the coating concentration required by β₃/MT-MCF7 cells (0.3-0.5 μg/ml vs. 0.05-0.06 μg/ml, respectively). Both cell lines were selected for maximal αᵥβ₃ expression by flow cytometry (FIG. 4, left panel) and express similar levels of integrin αᵥβ₃, as shown by immunoprecipitation (FIG. 5B), indicating that the two cell lines have a similar number of integrin αᵥβ₃ sites. In particular, mean fluorescence intensity after staining of β₃-MCF7 and ₃/MT-MCF7 cells with mAb LM609 was 38.7+/−5.6 and 35.5+/−7, respectively, a difference that was statistically not significant (t=0.896, p=0.394 at the 0.05 level in the paired Student test, n=11). These results indicate that the αᵥβ₃ integrin in the β₃/MT-MCF7 cells differs in its affinity for vitronectin as compared to the αᵥβ₃ integrin in β₃-MCF7 cells.

To determine the relative contribution of integrin αᵥβ₃ and MT1-MMP to cell motility, the efficiency of transfected cells in directional migration was evaluated in Transwells in which the membrane undersurface was coated with vitronectin. As shown in FIG. 8B, the migration of control MCF7 and MT-MCF7 cells was relatively modest, with only 3-6×10³ cells, or 6-12% of cells plated per well, transmigrating across the filter in 48 hours. Furthermore, β₃-MCF7 cells, which express integrin αᵥβ₃, also did not migrate in the Transwell assay. In six independent experiments, an average of 3.3+/−0.4×103 cells (n=18), or 5.8-7.4% of 50×10³ cells plated per well, migrated onto the vitronectin-coated membrane undersurface. In contrast, the migration efficiency of β₃/MT-MCF7 cells was substantially higher, with 23.3+/−2.1×103 cells (n=21), or 42.4-50.8% of cells plated per well, migrating to the vitronectin-coated membrane undersurface. These data corroborate the vitronectin-binding data disclosed above and demonstrate that the αᵥβ₃ integrins in β₃-MCF7 and β₃/MT-MCF7 cells are functionally distinct in their ability to mediate directional cell migration on vitronectin.

As further shown in FIG. 8C, the migration efficiency of β₃/MT-MCF7 cells far exceeded that of β₃-MCF7 cells in experiments where cells migrated in Transwells with the undersurface coated with increasing concentrations of vitronectin. While the migration of β₃-MCF7 cells was relatively low and did not increase proportionally relative to increasing concentrations of vitronectin, the migration of β₃/MT-MCF7 cells was dose-dependent in a concentration range from 0.1 to 5 μg/ml vitronectin. Taken together, these data demonstrate the functional activation of integrin αᵥβ₃ in β₃/MT-MCF7 cells.

MT1-MMP activity was examined relative to the functional activation of integrin αᵥβ₃ in ₃/MT-MCF7 cells. After first incubating β₃-MCF7 and β₃/MT-MCF7 cells for 18 hours in serum-free AIM-V medium, the cells were allowed to migrate in Transwells with and without Batimastat for 48 hours. As shown in FIG. 8D, Batimastat, if added directly to Transwells, did not significantly affect the migration of β₃/MT-MCF7 cells. However, if β3/MT-MCF7 cells were pre-incubated with Batimastat for 18 hours and then allowed to migrate in the presence of the inhibitor, cell migration was strongly inhibited. These results indicate that the functional activation of integrin αᵥβ₃ in β₃/MT-MCF7 cells is MT1-MMP-dependent.

In sum, these data indicate that MT1-MMP mediates the functional activation of integrin αᵥβ₃ in MCF7 cells and thereby facilitates the ligand-specific attachment and migration of these cells. These results further indicate that MT1-MMP and integrin avβ3 contribute jointly, but not individually, to the efficient directional locomotion of MCF7 cells.

The directional migration of cells in Transwells (Costar, Cambridge, Mass.) was analyzed under serum-free conditions (Deryugina et al., supra, (1997)) essentially as follows. The undersurface of a 6.5 mm insert membrane with a 8 micron pore size was coated with vitronectin. Cells detached with enzyme-free buffer (Specialty Media, Lavalette, N.J.) were plated into the insert at 5×10⁴ in 0.15 ml AIM-V medium. The outer chamber was filled with 0.5 ml of AIM-V medium. Following a 48 hour incubation, cells that migrated to the membrane's undersurface were detached with trypsin/EDTA and counted.

Adhesion assays were performed as follows using high binding 96-well plates (Corning, Corning, N.Y.) pre-coated with vitronectin (from 0.01 to 20 μg/ml), PEX (20 μg/ml) or mfAb LM609 (2 μg/ml) (Deryugina et al., supra, (1997); Deryugina et al., supra, (1998)). Cells (5×10⁴ cells per well in 0.1 ml of DMEM/1%BSA/20 mM HEPES buffer, pH 7.2) were allowed to bind to plastic coated with vitronectin or Inabs for 1 hour and with PEX for 8 hours at 37° C. in a CO₂-incubator. Function-blocking anti-integrin mabs were used at a final concentration of 25 μg/ml. Bound cells were stained with Crystal Violet. The incorporated dye was extracted with 100 mM sodium phosphate/50% ethanol, pH 4.5 before measuring absorbance at 540 nm.

### EXAMPLE IV

### Activated Integrin αᵥβ₃ Binds MMP-2 Via the PEX Domain

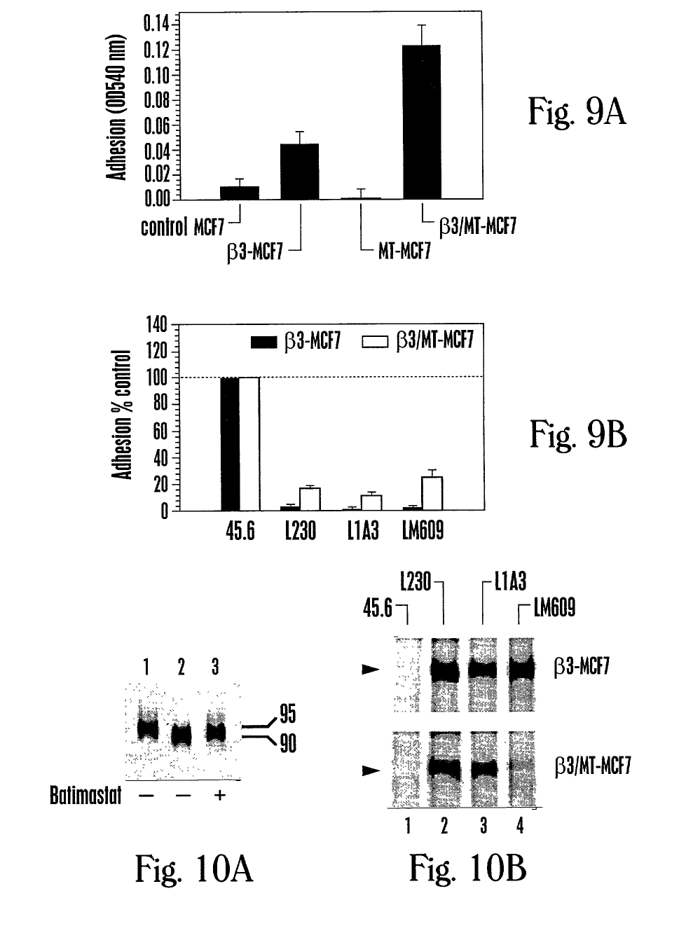

Integrin αᵥβ₃ binds the recombinant C-terminal domain of MMP-2 (PEX; Brooks et al., Cell 85:683-693 (1996)), allowing tumor cells to bind to PEX (Deryugina et al., supra, (1997)). The MT1-MMP-mediated activation of integrin αᵥβ₃ was analyzed relative to MMP-2 docking at cell surfaces by evaluating the adhesion of MCF7 cells to PEX. Cells were allowed to bind for 8 hours to PEX coated on plastic at 20 μg/ml. Whereas almost no binding to PEX was observed with control and MT-MCF7 cells, expression of the αᵥβ₃ integrin correlated with the ability of cells (β₃-MCF7 and β₃/MT-MCF7) to adhere to PEX. As shown in FIG. 9A, the adhesion efficiency of β₃/MT-MCF7 cells was substantially greater than the adhesion of 3-MCF7 cells. Cell adhesion was blocked by the αᵥβ₃-specific mAb LM609 and by two αv-specific mAbs, L230 and L1A3, confirming that integrin αᵥβ₃ mediates the binding of β₃-MCF7 and β₃/MT-MCF7 cells to PEX (see FIG. 9B). These findings indicate that the MT1-MMP-mediated activation of integrin αᵥβ₃ promotes the docking of MMP-2 via its C-terminal domain at the αᵥβ₃ cell surface sites. In turn, docking can facilitate activation of MMP-2 to the mature enzyme by cells simultaneously expressing integrin αᵥβ₃ and MT1-MMP.

### EXAMPLE V

### MT1-MMP-DEPENDENT Modifications of the β3 Integrin Subunit

Functional properties of activated integrin αᵥβ₃ were analyzed for a possible association with one or more structural modifications of β3 subunit. In particular, electrophoretic and immunological characteristics of the β3 integrin subunit from β₃-MCF7 and β₃/MT-MCF7 cells were compared by immunoprecipitation with mabs specific to αᵥ (L230 and L1A3) or αᵥβ₃ (LM609) integrins. As was shown in FIG. 5B, there is a 5 kDa difference in the molecular masses of the β3 integrin subunit precipitated from β₃-MCF7 and β₃/MT-MCF7 cells (FIG. 5B, lanes 2 and 4). These results indicate that MT1-MMP can mediate a proteolytic modification of the 3 integrin subunit.

To verify the proteolytic nature of modification of the 3 integrin subunit, β₃/MT-MCF7 cells were incubated with Batimastat to block metalloproteinase activity. As shown in FIG. 7C, Batimastat at a 50 μM concentration completely inhibited the MT1-MMP-induced activation of MMP-2 by β₃/MT-MCF7 cells. Without the inhibitor, the exogenously added 68 kDa pro-MMP-2 (FIG. 7C, lane 2) was efficiently converted into the 64 kDa intermediate and 62 kDa mature enzyme (FIG. 7C, lane 3). In contrast, in the presence of Batimastat, exogenous pro-MMP-2 remained a zymogen in β₃/MT-MCF7 cultures (FIG. 7C, lane 4), indicating that Batimastat completely inhibits MT1-MMP-dependent activation of MMP-2.

Furthermore, β₃-MCF7 and β₃/MT-MCF7 cells, incubated with and without 50 βM Batimastat for 48 hours, were surface labeled with biotin and lysed. Thereafter, αv integrins were immunoprecipitated from cell lysates with mAb L230 and analyzed by Western blotting. As shown in FIG. 10A, the apparent molecular mass of the β3 integrin subunit from β₃/MT-MCF7 cells incubated with Batimastat shifted to a 95 kDa value characteristic of the “wild-type” β3 from β₃-MCF7 cells (FIG. 10A, lanes 1 and 3). Thus, inhibition of MT1-MMP activity by Batimastat abolished the one or more modifications which account for the higher electrophoretic mobility of the 3 integrin subunit from β₃/MT-MCF7 cells.

To analyze whether MT1-MMP expression affects the immunological characteristics of the β3 integrin subunit, immunoprecipitation profiles of β3 precipitated from β₃-MCF7 and β₃/MT-MCF7 cells with mAbs L1A3 and L230, specific to the αᵥ integrin subunit, and mAb LM609 specific to integrin Uβ3, were compared. While the β3 integrin subunit was efficiently immunoprecipitated from β₃-MCF7 cells with all anti-integrin mAbs used, almost no 3 was observed in β₃/MT-MCF7 cells after precipitation with mAb LM609 (FIG. 10B). These findings indicate that the LM609 binding site of the β3 integrin subunit is modified in β₃/MT-MCF7 cells.

In sum, these data demonstrate that MT1-MMP mediates one or more modifications of the β3 subunit and that such modifications correlate with functional activation of integrin αᵥβ₃. These data further indicate that, by facilitating directional cell migration and MMP-2 binding at cell surfaces, functional activation of integrin αᵥβ₃ can be an important event in tumor invasion and metastasis.

### EXAMPLE VI

### Production and Characterization of a Constitutively Superactivated Form of the β3 Integrin

Production and Characterization of of a β3 Double Mutant

The β3 double mutant was prepared by PCR using the corresponding oligonucleotide primers. Mutagenesis to insert the N→T and L→Q substitutions at the position 69 and 70 of the β3 chain, respectively, were done by using the 204-GACTCAGCTGAAGGATAACTGTGCCCC-230 (SEQ ID NO: 11) direct primer and the 203-TCCTTCAGGTCACAGCGAGGTGAGCCC-177 (SEQ ID NO: 12) reverse primer (mutated positions shown by underlining in FIG. 3). The resulting mutant HindIII/XbaI fragment was recloned into pcDNA3-neo. MCF7 breast carcinoma cells were stably transfected with the recombinant pcDNA-3-neo plasmid carrying the double β3 mutant insert by standard methods. Stable transfectant clones were selected by flow cytometry employing integrin βvβ3 specific LM609 monoclonal antibody. Flow cytometry experiments were performed as described above in Example 1. FIG. 11 demonstrates FACS analysis of MCF7 breast carcinoma cells stably transfected with the double mutant β3 integrin subunit. I

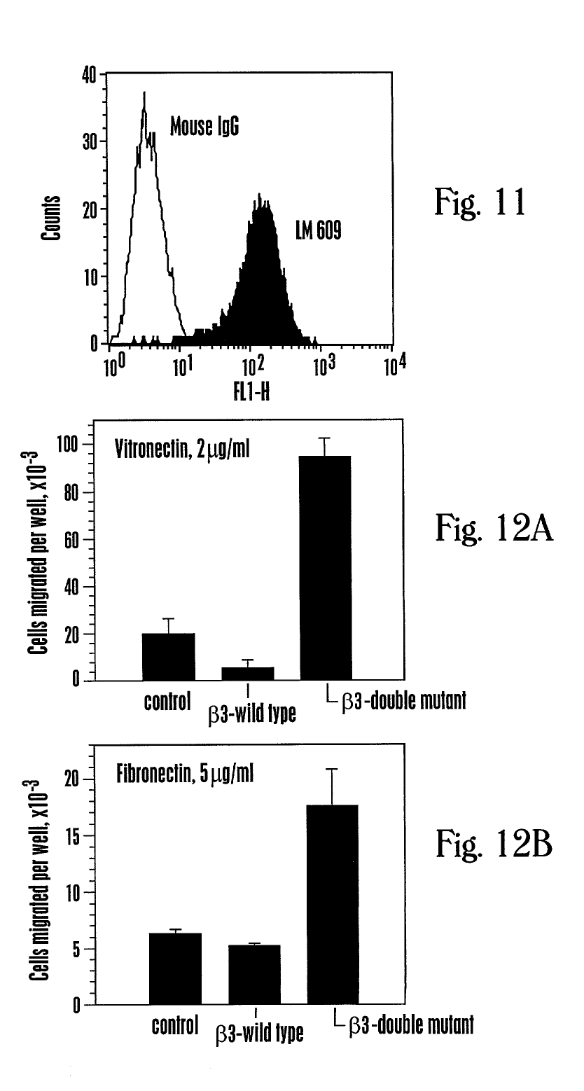

To determine the effect of mutation on cell motility, the efficiency of MCF7 cells transfected with the wild type and double mutant β3 subunit was evaluated in Transwells in which the membrane undersurface was coated with 2 μg/ml vitronectin and 5 μg/ml fibronectin (see FIG. 12). The migration assays were executed as described above in Example III. As shown in FIG. 12, migration of cells transfected with the double β3 mutant was significantly higher than that of control MCF7 cells and MCF7 cells transfected with the wild type β3 subunit.

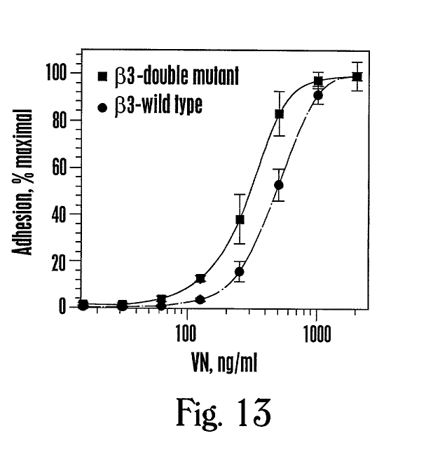

To evaluate the effect of mutation on cell adhesion, cells expressing the wild type and the double β3 mutant were allowed to adhere to plastic pre-coated with increasing concentrations of vitronectin. The experiments were performed under experimental conditions described in Example III. As shown in FIG. 13, cells expressing the mutant β3 integrin demonstrated greater adhesion efficiency relative to that of cells expressing the wild type β3 integrin. These findings indicate that the functional properties of the β3 double mutant correspond to those observed in cells doubly transfected with MTI-MMP and the wild type β3 integrin. Thus, integrin αᵥβ₃ bearing the double β3 mutant integrin subunit can be classified as a superactivated integrin exactly as integrin αᵥβ₃ modified by MT1-MMP in β3/MT-MCF7 doubly transfected cells.

### EXAMPLE VII

### Screening for Agonists and Antagonists of Superactivated αᵥβ₃

Doubly transfected β3/MT1-MMP-MCF7 cells expressing superactivated integrin αᵥβ₃ prepared as described above are utilized in the assay. Neo/zeo-MCF7 cells transfected with both neo-pcDNA3 and zeo-pcDNA3 plasmids with no inserts are used as the control. Prior to plating cells, individual chemicals in DMSO (1 ml) from corresponding stock solutions of the DIVERSet™ library (ChemBridge) are added directly to wells of a 96 well plate to a final concentration of 10-30 μM. Inhibition of cell adhesion identifies useful therapeutic candidates, which are re-assayed in the concentration range 0.1-10 μM. Since the half-life of αᵥβ₃ receptors on cell surfaces is about 24 hours (Delcommenne and Streuli, J. Biol. Chem. 270:26794-26801 (1995)), metabolic modifications do not affect the results of the one-hour assay.

Results are interpreted as follows. Reduction of adhesion of β3/MT1-MMP-MCF7 cells but not of control neo/zeo-MCF7 cells to vitronectin after a one hour incubation period identifies the library member as a specific αᵥβ₃ inhibitor. Little or no toxic effect is seen on control MCF7 cells for a specific αᵥβ₃ inhibitor.

Reduction in adhesion of both neo/zeo-MCF7 and β3/MT1-MMP- MCF7 cells is indicative of an indirect effect on the αᵥβ₃ a nonspecific effect on tumor cell membranes, or a toxic effect on tumor cells. Increased adhesion of β3/MT1-MMP-MCF7 upon treatment with a library member that does not affect adhesion of control neo/zeo-MCF7 cells identifies the library member as an enhancer of αᵥβ₃ functional activity. Such a compound can be useful, for example, in tissue regeneration.

Cell culture is performed as follows. β3/MT1-MMP-MCF7 and neo/zeo-MCF7 cells are maintained routinely in DMEM/FCS supplemented with the appropriate selective antibiotic (zeocin or G418). Prior to addition of :+library compounds to be assayed, cells are incubated in serum free media for 18 to 24 hours. Cells are harvested with an enzyme-free buffer, washed and re-suspended in serum-free DMEM or AIM-V media for subsequent assays.

Cell adhesion assays are performed in high binding 96-well plates (Corning) as follows. The wells are pre-coated with 0.1 μg/ml vitronectin in PBS (Deryugina et al., J. Cell Sci. 110:2473-2482 (1997) and Deryugina et al., Cancer Res. 58:3743-3750 (1998)), which is the concentration of vitronectin shown to be most efficient in discriminating αᵥβ₃-mediated cell adhesion relative to that of control cells (see, also, FIG. 8. Prior to addition of test compounds, the wells are blocked by addition of 1% BSA. Test compounds (1 μl in DMSO from the corresponding 10-200 μM stocks) are added to cells (5×10⁴ cells per well in 0.1 ml of DMEM/1%BSA/20 mM HEPES buffer, pH 7.2), which are incubated for 1 hour at 37° C. in a CO₂-incubator. Cells are stained with Crystal Violet to detect bound cells. The incorporated dye is extracted with 100 mM sodium phosphate/50% ethanol, pH 4.5, and absorbency measured at 540 nm. Cells are plated in the presence of 1% DMSO as a negative control. In addition, cells are treated with 1 mg/ml of RGD-peptide or 25 μg/ml of function blocking αᵥβ₃-specific LM609 rAb (Chemicon International, Temecula, Calif.) for use as positive controls. This assay also can be performed with 96-well plates pre-coated with other major individual extracellular matrix proteins such as collagen type I or IV, tenascin or laminin.

Cell adhesion assays also are performed on plastic coated with the αᵥβ₃-specific function-blocking monoclonal antibody, LM609. Plates are coated with anti-mouse polyclonal antibodies at 5-10 μg/ml, and subsequently with anti-αᵥβ₃ LM609 mAb at 0.5-1 μg/ml. Cell adhesion experiments then are performed as described above. Since only cells expressing integrin αᵥβ₃ are adhesive under these experimental conditions, this assay facilitates specific and direct identification of αᵥβ₃ antagonists by observing inhibition of adhesion of β3/MT1-MMP-MCF7 cells. The use of function-blocking αv- (clones NKI-M9 and AV1), β3- (clone B3A)- and αᵥβ₃-specific antibodies (clone P1F6; all clones from Chemicon International, Temecula, Calif.) also can be used to facilitate specific identification of αᵥβ₃ antagonists.

A standard chromium-51 release assay is used to evaluate cytotoxicity of inhibitors of superactivated αᵥβ₃. Cytotoxicity found in both control neo/zeo- and β3/MT1-MMP-MCF7 cells at an LD₅₀ that is 100 times the ID₅₀ for inhibition of adhesion to vitronectin signifies a non-integrin mediated nonspecific and undesirable effect. Cytotoxicity not found in control cells but found in β3/MT1-MMP-MCF7 cells indicates a specific integrin-mediated effect that can be apoptotic in nature. Further LD₅₀ experiments are performed under in vivo conditions.

### EXAMPLE VIII

### Further Characterization of Antagonists of Superactivated αᵥβ₃

Cell-based in Vitro Assays

If desired, antagonists of superactivated αᵥβ₃ can be further characterized using cell-based in vitro assays to determine their efficiency in blocking cell migration, invasion and proliferation.

Cell proliferation is assayed as follows using the ³H-thymidine incorporation method. Control neo/zeo- and β3/MT1-MMP-MCF7 cells are seeded into 96-well plates (3-5×10³ cells/well) and incubated with and without the antagonists of superactivated αᵥβ₃ in serum-containing and serum-free media. Following one to two days of incubation at 37° C., cell cultures are pulsed with 0.5 mCi/well of ³H-thymidine for the last 2 to 6 hours, and then washed and lysed. After transferring cell lysates onto glass filters, the incorporated ³H-thymidine is counted by standard methods.

A two-dimensional spheroid outgrowth assay employing control neo/zeo- and β3/MT1-MMP-MCF7 cells is performed with and without antagonists of superactivated UVβ3 on different ECM proteins, such as vitronectin, fibronectin, collagen type I and IV, tenascin and laminin substrates as described earlier (Deryugina et al., J. Cell Sci. 109:643-652 (1996)).

Cell migration (haptotactic) and Matrigel invasion assays are performed as follows. The migratory characteristics of control neo/zeo- and β3/MT1-MMP-MCF7 cells also are assessed in 6.5 mm Transwells (8 mm pore size), with the membrane undersurface coated with individual ECM proteins such as vitronectin, fibronectin, collagen type I or IV, tenascin or laminin. Cell invasion assays are performed in Transwells with the membrane pores occluded with Matrigel as described in Deryugina et al., Anticancer Res. 17:3201-3210 (1997).

Apoptotic effects of antagonists of superactivated αᵥβ₃ are evaluated using conventional methods involving BrdU, TUNEL and Annexin V in tissue cell cultures and by flow cytometric analyses.

MMP-2 binding activity of superactivated αᵥβ₃ treated with an antagonist is evaluated as follows. Superactivated αᵥβ₃ specifically binds MMP-2 via the C-terminal hemopexin-like (PEX) domain of MMP-2 (Deryugina et al., Int. J. Cancer 86:15-23 (2000); Brooks et al., Cell 92:391-400 (1998); and Brooks et al., Cell 85:683-693 (1996)). To evaluate whether the MMP-2 binding activity of superactivated αᵥβ₃ is affected by antagonists, β3- and β3/MT1-MMP-MCF7 cells expressing “wild-type” and “superactivated” αᵥβ₃, respectively, and non-adherent neo/zeo control cells (negative control) are assayed with and without antagonists by adhesion on plastic coated with recombinant PEX at 20 μg/ml as described previously (Deryugina et al., Int. J. Cancer 86:15-23 (2000) and Deryugina et al., J. Cell Sci. 110:2473-2482 (1997)).

Inhibitory Activity of Antagonists of Superactivated αᵥβ₃ Against other Integrins

If desired, antagonists of superactivated αᵥβ₃ are assayed for the ability to inhibit other integrins such as αᵥβ₅, αᵥβ₁ and αᵥβ₆ using cancer cell lines known to stably express high levels of these integrins. The human 293 embryonic kidney expresses αᵥβ₁ (Hu et al., J. Biol. Chem. 270:26232-26238 (1995)); SW480 colon adenocarcinoma cells express αᵥβ₆ (Agres et al., J. Cell Biol. 127:547-556 (1994)); available from Dr. R. Pytela); and U251 glioma cells express uV,5 (Deryugina et al., J. Cell Sci. 110:2473-2482 (1997) and Deryugina et al., Anticancer Res. 17:3201-3210 (1997)). The appropriate cells are plated on plastic coated with integrin-specific function-blocking antibodies (clone 6S6 for anti-β1; clone 10D5 for anti-αᵥβ₆ and clone P1F6 for αᵥβ₅ ; all clones are from Chemicon) and individual matrix proteins such as vitronectin, fibronectin, tenascin, collagen type I or IV at 0.1-10 βg/ml with and without the antagonists of superactivated αᵥβ₃. Function-blocking αᵥ specific L230 nab (ATCC) or LiAL mAb (Deryugina et al., Int. J. Cancer 86:15-23 (2000)) or RGD-peptides are used as controls. Adherent cells are counted as described above.

Use of Xenoaraph Models in Immuno-deficient Mice to Analyze Anti-tumoricfenic Activity of Antagonists of αᵥβ₃

In vivo anti-tumorigenic and anti-angiogenic effects of antagonists of superactivated αᵥβ₃ can be demonstrated as follows.

To evaluate tumor growth and metastatic potential, β3/MT1-MMP-MCF7 and MT1-MMP-U251.3 human glioma cells are injected into the mammary fat pads and in the tail vein of 6 week old female nu/nu mice, respectively (5×10⁶ cells per site; 5-8 mice/group). Tumors originating from MT1-MMP-U251.3 cells have previously been characterized as having very high growth rates and high levels of neovascularization relative to control cells. The use of transfected MT1-MMP-U251.3 glioma cells facilitates evaluation of in vivo anti-tumorigenic effects of antagonists of superactivated αᵥβ₃. These antagonists are administered i.p. 1, 3 and 10 mg/kg body weight daily for 6-8 weeks (four groups including control), and their effects on tumor growth, metastasis and neovascularization evaluated.

Tumor growth is monitored weekly by measuring mean diameter of the tumors. Size of the tumors is estimated as a volume of a sphere with the mean diameter of the tumor (mm³). At 6-8 weeks mice are sacrificed by cervical dislocation, and tumors are resected, weighed and dissected. For morphological and immunohistochemical analyses, tumor sections are frozen in liquid nitrogen or fixed.

To assess metastatic potential of cell transfectants, lungs and kidneys from the euthanized mice are examined for tumor nodules. Before euthanasia, Evans blue is injected intravenously, followed by vascular perfusion. Tumor growth in the mammary fat pads is evaluated weekly, and sections from resected tissues are analyzed by immunohistochemistry as described below.

Immunohistochemistry is performed as follows. Primary tumors and internal organs of interest (lungs, kidneys, liver) from nu/nu mice are resected and frozen or embedded in paraffin. Sections (5-6 mm thick) are prepared and stained with 10-20 mg/ml of MT1-MMP-, αᵥ, αᵥβ₃-, β3- and MMP-2-specific antibodies, which are commercially available from Calbiochem, Chemicon International, Neomarkers and Amersham. Bound primary antibodies are detected with the appropriate secondary antibodies conjugated with HRP. Peroxidase activity in sections is developed with DAB. To measure levels of MMP-2 and MT1-MMP, resected tissues are extracted with 2% SDS, and the extracts analyzed by Western blotting and zymography. CD31 staining is used to verify levels of tumor angiogenesis.

MMP-2 activity is analyzed as follows. Since MT1-MMP initiates activation of MMP-2 and integrin αᵥβ₃ facilitates activation to the MMP-2's maturation (Deryugina et al., Int. J. Cancer 86:15-23 (2000)), MMP-2 activity is analyzed in gels co-polymerized with 0.1% gelatin. For these purposes, the core (most central) and periphery (adjacent to the capsule) portions of each tumor are extracted overnight with 2×SDS sample buffer (1:4, w/v) at room temperature. The extracts are mixed 1:1 with 60% glycerol, analyzed and processed by gelatin zymography as described in Deryugina et al., J. Cell Sci. 110:2473-2482 (1997).

Optimization of Antagonists of αᵥβ₃

Optimization of agonists and antagonists of superactivated αᵥβ₃ is performed as follows. A computerized search by using Chemix (Oxford Molecular Group, Oxford, UK) and ISIS (MDL Information Systems, San Leandro, Calif.) software is made in order to identify other 2-D and 3-D structurally related compounds in libraries such as ChemBridge libraries. Such structurally related molecules are screened for the ability to specifically inhibit superactivated αᵥβ₃, and optimized inhibitors are re-tested in vitro and in vivo. Further, cytotoxicity and the LD₅₀ value of optimized compounds is assessed by standard methods.

All journal article, reference, and patent citations provided above, in parentheses or otherwise, whether previously stated or not, are incorporated herein by reference.

Although the invention has been described with reference to the examples above, it should be understood that various modifications can be made without departing from the spirit of the invention. Accordingly, the invention is limited only by the following claims.

We claim:

1. A method of identifying an inhibitor or enhancer of αᵥβ₃ activity, comprising the steps of:
(a) contacting superactivated αᵥβ₃ integrin with one or more molecules; and
(b) assaying an αᵥβ₃ integrin activity,
wherein reduced αᵥβ₃ activity identifies an inhibitor of αᵥβ₃ activity,
and wherein enhanced αᵥβ₃ activity identifies an enhancer of αᵥβ₃ activity.

2. The method of claim 1, wherein said αᵥβ₃ integrin activity is reduced activity.

3. The method of claim 1, wherein said αᵥβ₃ integrin activity is enhanced activity.

4. The method of claim 1, wherein said superactivated αᵥβ₃ integrin is expressed on a cell.

5. The method of claim 4, wherein said cell is a tumor cell.

6. The method of claim 4, wherein said cell is an immortalized cell.

7. The method of claim 4, wherein said cell is a MCF-7 breast carcinoma cell.

8. The method of claim 4, wherein said cell is transfected with a β3 encoding nucleic acid molecule and an MT1-MMP encoding nucleic acid molecule.

9. The method of claim 8, wherein said β3 has substantially the amino acid sequence of SEQ ID NO: 2 and said MT1-MMP has substantially the amino acid sequence of SEQ ID NO: 4.

10. The method of claim 9, wherein said cell is a MCF-7 breast carcinoma cell.

11. The method of claim 4, wherein said cell is transfected with a nucleic acid molecule encoding a superactivated β3 variant.

12. The method of claim 11, wherein said superactivated β3 variant has substantially the amino acid sequence of SEQ ID NO: 6.

13. The method of claim 12, wherein said cell is a MCF-7 breast carcinoma cell.

14. The method of claim 1, wherein said αᵥβ₃ integrin activity is cell adhesion activity.

15. The method of claim 14, wherein said αᵥβ₃ integrin activity is vitronectin-binding activity.

16. The method of claim 14, wherein said αᵥβ₃ integrin activity is fibronectin-binding activity.

17. The method of claim 14, wherein said αᵥβ₃ integrin activity is adhesion to a function blocking αᵥβ₃-specific antibody.

18. A superactivated β3 variant, comprising substantially the amino acid sequence of a β3 subunit with a threonine analog at the equivalent of position 69 and a glutamine analog at the equivalent of position 70,
wherein, when expressed together with an βᵥ subunit, said β3 variant forms superactivated αᵥβ₃ integrin in the absence of MT1-MMP.

19. The superactivated β3 variant of claim 18, comprising a threonine at the equivalent of position 69 and a glutamine at the equivalent of position 70.

20. The superactivated β3 variant of claim 18, comprising substantially the amino acid sequence of SEQ ID NO: 6.

21. The superactivated β3 variant of claim 20, comprising the amino acid sequence SEQ ID NO: 6.


SEQUENCE LISTING (12 sequences)

SEQ ID NO: 1 - DNA, 2367 bp, Homo sapiens
  CDS (1)...(2364)
atgcgagcgcggccgcggccccggccgctctgggtgactgtgctggcgctgggggcgctggcgggcgttggcgtaggagggcccaacatctgtaccacgcgaggtgtgagctcctgccagcagtgcctggctgtgagccccatgtgtgcctggtgctctgatgaggccctgcctctgggctcacctcgctgtgacctgaaggagaatctgctgaaggataactgtgccccagaatccatcgagttcccagtgagtgaggcccgagtactagaggacaggcccctcagcgacaagggctctggagacagctcccaggtcactcaagtcagtccccagaggattgcactccggctccggccagatgattcgaagaatttctccatccaagtgcggcaggtggaggattaccctgtggacatctactacttgatggacctgtcttactccatgaaggatgatctgtggagcatccagaacctgggtaccaagctggccacccagatgcgaaagctcaccagtaacctgcggattggcttcggggcatttgtggacaagcctgtgtcaccatacatgtatatctccccaccagaggccctcgaaaacccctgctatgatatgaagaccacctgcttgcccatgtttggctacaaacacgtgctgacgctaactgaccaggtgacccgcttcaatgaggaagtgaagaagcagagtgtgtcacggaaccgagatgccccagagggtggctttgatgccatcatgcaggctacagtctgtgatgaaaagattggctggaggaatgatgcatcccacttgctggtgtttaccactgatgccaagactcatatagcattggacggaaggctggcaggcattgtccagcctaatgacgggcagtgtcatgttggtagtgacaatcattactctgcctccactaccatggattatccctctttggggctgatgactgagaagctatcccagaaaaacatcaatttgatctttgcagtgactgaaaatgtagtcaatctctatcagaactatagtgagctcatcccagggaccacagttggggttctgtccatggattccagcaatgtcctccagctcattgttgatgcttatgggaaaatccgttctaaagtcgagctggaagtgcgtgacctccctgaagagttgtctctatccttcaatgccacctgcctcaacaatgaggtcatccctggcctcaagtcttgtatgggactcaagattggagacacggtgagcttcagcattgaggccaaggtgcgaggctgtccccaggagaaggagaagtcctttaccataaagcccgtgggcttcaaggacagcctgatcgtccaggtcacctttgattgtgactgtgcctgccaggcccaagctgaacctaatagccatcgctgcaacaatggcaatgggacctttgagtgtggggtatgccgttgtgggcctggctggctgggatcccagtgtgagtgctcagaggaggactatcgcccttcccagcaggacgagtgcagcccccgagagggtcagcccgtctgcagccagcggggcgagtgcctctgtggtcaatgtgtctgccacagcagtgactttggcaagatcacgggcaagtactgcgagtgtgacgacttctcctgtgtccgctacaagggggagatgtgctcaggccatggccagtgcagctgtggggactgcctgtgtgactccgactggaccggctactactgcaactgtaccacgcgtactgacacctgcatgtccagcaatgggctgctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccgggagccctacatgaccgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtagaagagccagagtgtcccaagggccctgacatcctggtggtcctgctctcagtgatgggggccattctgctcattggccttgccgccctgctcatctggaaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaa

SEQ ID NO: 2 - protein, 788 aa, Homo sapiens [sequence omitted]

SEQ ID NO: 3 - DNA, 1749 bp, Homo sapiens
  CDS (1)...(1746)
atgtctcccgccccaagacccccccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaatcactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccaggccccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgctgcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctga

SEQ ID NO: 4 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 5 - DNA, 2367 bp, Homo sapiens
  CDS (1)...(2364)
atgcgagcgcggccgcggccccggccgctctgggtgactgtgctggcgctgggggcgctggcgggcgttggcgtaggagggcccaacatctgtaccacgcgaggtgtgagctcctgccagcagtgcctggctgtgagccccatgtgtgcctggtgctctgatgaggccctgcctctgggctcacctcgctgtgacctgaaggagactcagctgaaggataactgtgccccagaatccatcgagttcccagtgagtgaggcccgagtactagaggacaggcccctcagcgacaagggctctggagacagctcccaggtcactcaagtcagtccccagaggattgcactccggctccggccagatgattcgaagaatttctccatccaagtgcggcaggtggaggattaccctgtggacatctactacttgatggacctgtcttactccatgaaggatgatctgtggagcatccagaacctgggtaccaagctggccacccagatgcgaaagctcaccagtaacctgcggattggcttcggggcatttgtggacaagcctgtgtcaccatacatgtatatctccccaccagaggccctcgaaaacccctgctatgatatgaagaccacctgcttgcccatgtttggctacaaacacgtgctgacgctaactgaccaggtgacccgcttcaatgaggaagtgaagaagcagagtgtgtcacggaaccgagatgccccagagggtggctttgatgccatcatgcaggctacagtctgtgatgaaaagattggctggaggaatgatgcatcccacttgctggtgtttaccactgatgccaagactcatatagcattggacggaaggctggcaggcattgtccagcctaatgacgggcagtgtcatgttggtagtgacaatcattactctgcctccactaccatggattatccctctttggggctgatgactgagaagctatcccagaaaaacatcaatttgatctttgcagtgactgaaaatgtagtcaatctctatcagaactatagtgagctcatcccagggaccacagttggggttctgtccatggattccagcaatgtcctccagctcattgttgatgcttatgggaaaatccgttctaaagtcgagctggaagtgcgtgacctccctgaagagttgtctctatccttcaatgccacctgcctcaacaatgaggtcatccctggcctcaagtcttgtatgggactcaagattggagacacggtgagcttcagcattgaggccaaggtgcgaggctgtccccaggagaaggagaagtcctttaccataaagcccgtgggcttcaaggacagcctgatcgtccaggtcacctttgattgtgactgtgcctgccaggcccaagctgaacctaatagccatcgctgcaacaatggcaatgggacctttgagtgtggggtatgccgttgtgggcctggctggctgggatcccagtgtgagtgctcagaggaggactatcgcccttcccagcaggacgagtgcagcccccgagagggtcagcccgtctgcagccagcggggcgagtgcctctgtggtcaatgtgtctgccacagcagtgactttggcaagatcacgggcaagtactgcgagtgtgacgacttctcctgtgtccgctacaagggggagatgtgctcaggccatggccagtgcagctgtggggactgcctgtgtgactccgactggaccggctactactgcaactgtaccacgcgtactgacacctgcatgtccagcaatgggctgctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccgggagccctacatgaccgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtagaagagccagagtgtcccaagggccctgacatcctggtggtcctgctctcagtgatgggggccattctgctcattggccttgccgccctgctcatctggaaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaa

SEQ ID NO: 6 - protein, 788 aa, Homo sapiens
MRARPRPRPLWVTVLALGALAGVGVGGPNICTTRGVSSCQQCLAVSPMCAWCSDEALPLGSPRCDLKETQLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDREPYMTENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 7 - protein, 7 aa, Artificial Sequence
  synthetic construct
PRCGXPD

SEQ ID NO: 8 - protein, 4 aa, Artificial Sequence
  synthetic construct
RXXR

SEQ ID NO: 9 - protein, 11 aa, Artificial Sequence
  synthetic construct
HEXXHXXGXXH

SEQ ID NO: 10 - protein, 4 aa, Artificial Sequence
  synthetic construct
RRKR

SEQ ID NO: 11 - DNA, 27 bp, Homo sapiens
gactcagctgaaggataactgtgcccc

SEQ ID NO: 12 - DNA, 27 bp, Homo sapiens
tccttcaggtcacagcgaggtgagccc